(19) United States
(12) Patent Application Publication
(10) Pub. No.: US 2025/0281646 A1
(43) Pub. Date: Sep. 11, 2025

(54) PLASMID DNA CONSTRUCTS FOR THERAPEUTIC PROTEIN EXPRESSION

(71) Applicant: RenBio, Inc., Long Island City, NY (US)

(72) Inventors: Rachel LIBERATORE, Long Island City, NY (US); Neal PADTE, Long Island City, NY (US); Xin YAO, Long Island City, NY (US)

(21) Appl. No.: 18/862,796

(22) PCT Filed: May 5, 2023
(86) PCT No.: PCT/US2023/066688

§ 371 Date: Nov. 4, 2024

Related U.S. Application Data
(60) Provisional application No. 63/338,753, filed on May 5, 2022.
(60) Provisional application No. 63/338,774, filed on May 5, 2022.

(51) Int. Cl. A61K 48/00; C12N 15/85

(52) U.S. Cl. CPC A61K 48/0058; C12N 15/85; C12N 2800/107; C12N 2820/60

(57) ABSTRACT

The disclosure is directed to compositions that comprise a plasmid DNA construct having a DNA sequencing encoding a therapeutic protein, or a fragment thereof, in vivo, along with methods of generating and manufacturing the antibody or therapeutic protein, as well as methods for preventing and/or treating a disease in a patient.

### PRIORITY

This application claims the benefit of, and claims priority to, U.S. Provisional Application No. 63/338,753, filed May 5, 2022, and U.S. Provisional Application No. 63/338,774, filed May 5, 2022 which are each hereby incorporated by reference in their entireties.

### FIELD OF THE DISCLOSURE

The present disclosure relates to, in part, DNA compositions comprising expression constructs for producing a therapeutic protein in vivo, and methods for treating or preventing disease, including but not limited infectious diseases, inflammatory diseases, metabolic diseases, and cancer.

### DESCRIPTION OF THE TEXT FILE SUBMITTED ELECTRONICALLY

This application contains a Sequence Listing in ASCII format submitted electronically herewith via EFS-Web. Said ASCII copy, created on May 5, 2022, is named RBF-001PR_SequenceListing_ST25.txt and is 13,043 bytes in size. The Sequence Listing is incorporated herein by reference in its entirety.

### BACKGROUND

While protein and peptide therapeutics are widely used, e.g., for treatment and prevention of infectious disease, treatment of chronic inflammatory diseases and metabolic diseases, inheritable diseases, and for treatment of cancer, conventional protein drugs generally require repeated (often frequent) administrations, and can suffer from short half-life in the circulation. As a result, it is difficult to maintain conventional protein drugs within their therapeutic window for long periods of time, thereby leading to inconvenient dosing schedules, ineffective therapy, and adverse side effects. In the various aspects and embodiments, the present disclosure addresses these and other problems.

### SUMMARY

The present invention in various aspects and embodiments provides DNA constructs encoding therapeutic proteins for sustained and durable expression in a mammalian host, and methods for treating or preventing disease. The plasmid DNA constructs disclosed herein allow for production of therapeutic proteins (including antibodies) inside a patient's cell, such as a skeletal muscle cell. Such a production process allows for a muscle cell to act as an in vivo bioreactor in the expression and production of the therapeutic protein. In addition to manufacturing advantages compared to conventional protein therapy, the administration of the DNA constructs disclosed herein can result in a significant decrease in administration frequency (compared to protein administration) and maintain the circulating level of the therapeutic protein within a therapeutic window for a long duration. Accordingly, the plasmid DNA constructs disclosed herein allow for the treatment or prevention of numerous diseases and conditions using the patient's own cells for the steady production of the therapeutic protein over time.

In one aspect, the invention provides a DNA composition comprising a plasmid construct having at least one expression cassette, wherein the expression cassette comprises in the following order from 5′ to 3′: a) a CMV IE enhancer sequence: b) a chicken beta-actin promoter sequence: c) a CMV IE intron A sequence: d) a cloning site or an open reading frame encoding a therapeutic protein; and e) a transcription termination sequence. In some embodiments, the termination sequence is an artificial transcription termination sequence comprising segments of a bovine growth hormone polyadenylation signal (BGHpA).

The expression platform described herein can be used to deliver numerous therapeutic proteins, including antibodies, protein or peptide hormones, cytokines, and enzymes (e.g., for enzyme replacement therapy). In some embodiments, the therapeutic protein is an antibody heavy and/or antibody light chain. The antibody in some embodiments binds to and neutralizes a virus or an inflammatory cytokine, or targets a cancer cell, as illustrated by certain embodiments of this disclosure. In some embodiments, the virus is Zika virus, an influenza virus, a beta coronavirus, human immunodeficiency virus (HIV), hepatitis virus, a herpes virus, Epstein-Barr virus, or CMV, or other virus disclosed herein. In some embodiments, the antibody (or other therapeutic protein, such as a soluble cytokine receptor) targets and neutralizes the action of a pro-inflammatory cytokine selected from TNF-alpha, IL-1, IL-4, IL-6. IL-12, and IL-23. In still other embodiments, the antibody or therapeutic protein targets a cancer antigen selected from HER2, PSA, TRP-2, EpCAM, GPC3, mesothelin (MSLN), and EGFR.

In some embodiments, the therapeutic protein is a single chain variable fragment (scFv). In other embodiments, the plasmid construct comprises two of said expression cassettes, with a first expression cassette having an open reading frame encoding an antibody heavy chain and a second expression cassette comprising an open reading frame encoding antibody light chain, thereby ensuring that both chains are delivered and expressed in the same cell.

In still other embodiments, the open reading frame encodes antibody heavy and light chains with a peptide linker therebetween that induces ribosomal skipping (e.g., co-translational cleavage). Such peptide linkers may comprise P2A peptide or T2A peptide. The peptide linker may further comprise a furin recognition site on the N-terminal side of the peptide that induces ribosomal skipping, to ensure cleavage of remaining amino acids of P2A or T2A from the upstream protein. The linker sequence may further comprise a Gly Ser linker or other linker to ensure efficient processing.

In some embodiments, the expression cassette comprises the nucleotide sequence of SEQ ID NO: 1, where a therapeutic protein open reading frame can be inserted after the CMV IE intron A sequence.

In some embodiments, the expression cassette comprises the nucleotide sequence of SEQ ID NO: 3, where a therapeutic protein coding sequence can be inserted after the CMV IE intron A sequence.

In some embodiments, the expression cassette comprises the nucleotide sequence of SEQ ID NO: 5 or SEQ ID NO: 8, where a therapeutic protein coding sequence inserted on either side of the Furin T2A sequence, or wherein the therapeutic protein coding sequence is inserted on either side of a P2A sequence.

Exemplary embodiments provided by this disclosure express a guselkumab heavy and/or light chain, and may express the amino acid sequence of SEQ ID NO: 13 or SEQ ID NO: 14. Exemplary embodiments provided by this disclosure express a ustekinumab heavy and/or light chain, and may express the amino acid sequence of SEQ ID NO: 17 or SEQ ID NO: 18. Exemplary embodiments provided by this disclosure express a risankizumab heavy and/or light chain, and may express the amino acid sequence of SEQ ID NO: 21 or SEQ ID NO: 22.

In still other embodiments, the therapeutic protein is selected from a granulocyte colony stimulating factor, an erythropoietin, and an insulin-like growth factor. For example, the therapeutic protein may be filgrastim or epoetin alfa. In some embodiments, the therapeutic protein is a GLP-1 receptor agonist, a GIP receptor agonist, and/or a glucagon receptor agonist, including a dual or triple agonists. The therapeutic proteins and peptides may be encoded with albumin or Ig Fc fusion for half-life enhancement.

In other aspects, the invention provides a method for treating or preventing a viral infection. In these aspects the method comprises administering an effective amount of the composition according to the disclosure to a subject in need thereof. In this aspect, the antibody binds to and neutralizes the target virus. In this aspect, the subject may have a viral infection, or may be at risk of acquiring a viral infection.

Exemplary viruses that can be treated or prevented in various embodiments are selected from Zika virus, an influenza virus (A or B), a beta coronavirus, human immunodeficiency virus (HIV), hepatitis virus, a herpes virus, Epstein-Barr virus, and CMV, or other virus disclosed herein. In according with such embodiments, antibody heavy and light chains can be co-expressed as described herein using constructs with dual expression cassettes, or by cloning the heavy and light chains in frame with a ribosomal skipping peptide (e.g., P2A or T2A).

In other aspects, the disclosure provides a method for treating or preventing an inflammatory or autoimmune disease or disorder. The method comprises administering an effective amount of the composition of the disclosure to a subject in need thereof. In some embodiments, the therapeutic agent (such as an antibody) targets and neutralizes the action of IL-23. These embodiments are useful for treating, for example, a subject having plaque psoriasis or Crohn's disease. In some embodiments, the DNA construct may encode guselkumab, ustekinumab, or risankizumab, as disclosed herein. In other embodiments, the subject has arthritis, and the therapeutic protein may bind to and neutralize, for example, TNF-a or IL-6.

In other aspects, the disclosure provides a method for treating or preventing cancer, comprising administering an effective amount of the DNA composition of this disclosure to a subject in need thereof. For example, the subject may have stage 1, stage 2, stage 3, or stage 4 cancer. In some embodiments, the cancer is a solid tumor, and the DNA construct is administered following tumor resection. In some embodiments, he DNA construct is administered with or following chemotherapy, radiation therapy, or cell therapy that is optionally a T cell therapy (e.g., a CAR-T therapy). In some embodiments, the cancer is a blood cancer.

In other aspects, the disclosure provides a method for treating or preventing an inflammatory eye disease. The method comprises administering an effective amount of the composition of the disclosure. In some embodiments, the inflammatory eye disease is selected from an inflammatory eye disease associated with corneal transplant, diabetic macular edema, diabetic retinopathy, dry eye disease, scleritis, blepharitis, keratitis, conjunctivitis, chorioretinal inflammation, chorioretinitis, iridocyclitis, iritis, posterior cyclitis, and uveitis. The inflammatory eye disease may be is associated with a corneal allograft, or a corneal allograft rejection. In some embodiments, the inflammatory eye disease is uveitis which is anterior uveitis, panuveitis, intermediate uveitis or posterior uveitis. In some embodiments, the therapeutic agent may bind to and neutralize a pro-inflammatory cytokine.

In other aspects, the disclosure provides a method for improving a patient response to allogeneic hematopoietic stem cell transplantation (aHSCT). The method comprises administering an effective amount of the composition of the disclosure to a subject in need. For example, the therapeutic agent may bind to and neutralize a pro-inflammatory cytokine.

In other aspects, the disclosure provides a method for treating chronic neutropenia. The method comprises administering an effective amount of the composition of the disclosure to a subject in need, where the DNA construct expresses G-CSF.

In other aspects, the disclosure provides a method for treating anemia. The method comprises administering an effective amount of the composition of the disclosure to a subject in need, wherein the DNA construct expresses erythropoietin.

In other aspects, the disclosure provides a method for treating a rare disease. The method comprises administering an effective amount of the composition of the disclosure to a subject in need thereof. In various embodiments, the rare disease involves an enzyme deficiency, and the DNA construct provides enzyme replacement therapy.

In the various aspects and embodiments, administering can comprise injection and electroporation. In various embodiments, the administering is intramuscular injection. In some embodiments, the antibody is expressed in the muscle cell and released into the circulation of the subject. In some embodiments, the method further comprises monitoring the presence of the antibody in the circulation at least once.

Other aspects and embodiments of the invention will be apparent from the following detailed description and working examples.

### BRIEF DESCRIPTION OF THE DRAWINGS

FIG. 1 is an image showing delivering of a therapeutic protein-encoding plasmid (pDNA) into a muscle cell, followed by expression and production of the antibody or therapeutic protein in the muscle cell, and uptake of the antibody or therapeutic protein in the circulation.

FIG. 2 shows an image of one of the hurdles facing protein therapeutics, as protein therapeutics encounter a significant dilutional effect as the blood volume increase from e.g., a rabbit to a human.

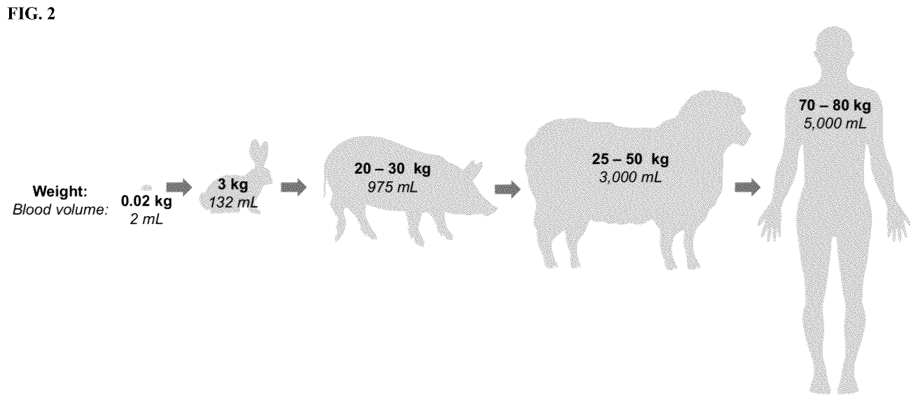

FIG. 3 is an image showing the lengthy time and cost consuming process for manufacturing proteins, antibodies, and drug-related products by current methods in the field.

FIG. 4A, FIG. 4B, FIG. 4C, and FIG. 4D are graphs demonstrating the application of the plasmid DNA constructs disclosed herein to a diverse set of disease indications in mouse disease models. FIG. 4A shows intramuscular delivery of a construct encoding an anti-HER2 antibody in a mouse model of breast cancer. FIG. 4B shows intramuscular delivery of a construct encoding an anti-TNFα antibody in a mouse model of arthritis. FIG. 4C shows intramuscular delivery of a construct encoding an anti-Influenza A antibody in a mouse model for treatment of Flu. FIG. 4D shows intramuscular delivery of a construct encoding EPO in a mouse model for treatment of anemia.

FIG. 5 is a non-limiting image showing plasmid DNA constructs according to embodiments of the disclosure.

FIG. 6 shows two graphs, labelled 1 and 2, demonstrating in vivo expression level of antibodies (both heavy and light chains) in mice using various plasmid constructs according to the disclosure.

FIG. 7 is a graph comparing CMV and CBA promoters in the plasmid constructs according to the present disclosure.

FIG. 8 is a graph showing the durability of protein expression (mouse anti-Influenza A antibody) over a long time course (i.e., at least 1 year).

FIG. 9 shows a series of non-limiting diagrams of DNA constructs expressing a therapeutic protein according to embodiments of the disclosure.

FIG. 10 is a graph showing the serum antibody concentration levels over a period of 1 year for different DNA constructs encoding the herceptin 4D5 antibody.

FIG. 11 shows a series of non-limiting diagrams of DNA constructs expressing a therapeutic protein according to embodiments of the disclosure.

FIG. 12 is a graph showing the serum antibody concentration levels over a period of 1 year for different DNA constructs encoding a therapeutic protein (s139 IgG).

FIG. 13 shows a series of non-limiting diagrams of DNA constructs expressing a therapeutic protein according to embodiments of the disclosure.

FIG. 14A, FIG. 14B, FIG. 14C, and FIG. 14D are graphs showing the levels of a therapeutic protein (s139 IgG) expressed in mice from different DNA constructs.

### DETAILED DESCRIPTION

The present invention in various aspects and embodiments provides DNA constructs encoding therapeutic proteins for sustained and durable expression in a mammalian host, and methods for treating or preventing disease. The plasmid DNA constructs disclosed herein allow for production of therapeutic proteins (including antibodies) inside a patient's cell (e.g., a muscle cell (e.g., a myocyte), such as a skeletal muscle cell, a cardiac muscle, and/or a smooth muscle cell). Such a production process allows for a muscle cell to act as an in vivo bioreactor in the expression and production of the therapeutic protein. The plasmid DNA constructs disclosed herein lack cold-chain storage requirements, and allow for the simultaneous expression and production of multiple polypeptides in vivo, and compared to conventional protein therapeutic delivery, the administration of the DNA constructs disclosed herein can result in a significant decrease in administration frequency and maintain the circulating level of the therapeutic protein within a therapeutic window for a long duration (e.g., at least 4 months, or at least 6 months, or at least 12 months, or at least 18 months, or at least 24 months). Accordingly, the plasmid DNA constructs disclosed herein allow for the treatment or prevention of numerous diseases and conditions using the patient's own cells for the steady production of the therapeutic protein over time.

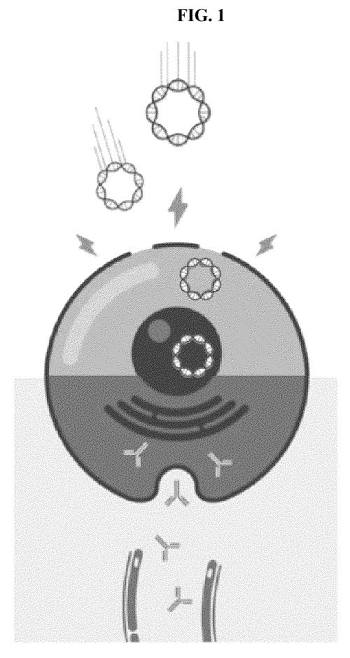

An overview of the plasmid DNA construct technology and delivery system, as disclosed herein, is shown in FIG. 1. This figure shows the intramuscular injection of a plasmid DNA construct having an expression cassette encoding a therapeutic protein as described herein. The plasmid DNA construct is delivered to muscle cells, allowing for steady expression and production of the therapeutic protein by the muscle cell. The antibody or therapeutic protein moves into peripheral circulation, similar to a conventional therapeutic protein administered intravenously or subcutaneously.

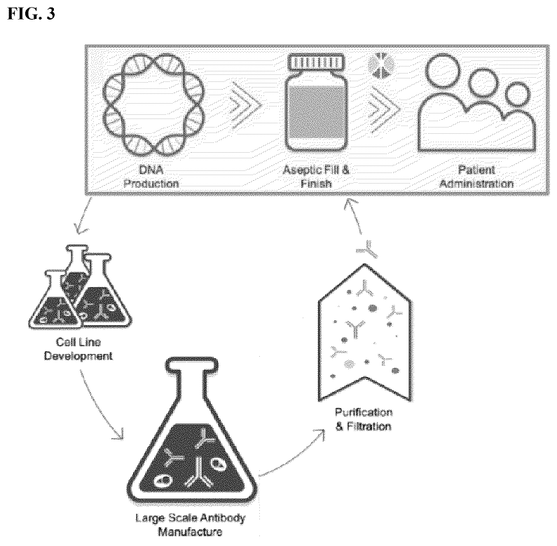

In addition to the above, the plasmid DNA construct technology described herein overcomes many of the roadblocks relevant to typical antibody and therapeutic protein manufacturing and development, which are shown in FIG. 3. The plasmid DNA construct, as described herein, significantly reduces the timeline for manufacturing, because no lengthy scale-up process is needed (FIG. 3), since the antibody or therapeutic protein is expressed and produced in the patient's own muscle cell (FIG. 1). The manufacturing timeline and production costs are significantly reduced, giving a significant advantage of the presently described invention over current methods of antibody and/or protein expression, production, and manufacturing. Further, the invention in certain embodiments, allows for one or multiple therapeutic proteins to be produced in the patient in a sustained and durable manner, to thereby avoid the large fluxes in circulating levels of therapeutic proteins often associated with conventional protein therapy, and/or to avoid frequent administrations of therapeutic compositions.

### Plasmid Design

The DNA compositions disclosed herein comprise a plasmid DNA construct, wherein the plasmid DNA construct comprises at least one expression cassette having the following elements in order of 5′ to 3′: a) an enhancer sequence: b) a promoter sequence: c) an intron sequence: d) a cloning site or an open reading frame encoding a therapeutic protein; and e) a transcription termination sequence. In some embodiments, the enhancer, promoter, and intron sequences are selected to provide for enhanced expression of the therapeutic protein in mammalian cells (including duration of expression), such as in muscle cells. In some embodiments, the enhancer is, for example, human cytomegalovirus (CMV) immediate-early enhancer. In some embodiments, the promoter is chicken beta-actin promoter. In some embodiments, the intron sequence is, for example, CMV IE intron A (or a truncated derivative thereof).

In some embodiments, the plasmid construct comprises two expression cassettes, where a first expression cassette has an open reading frame encoding a first protein (such as an antibody heavy chain) and a second expression cassette has an open reading frame encoding a second protein (which can be the same or different from the first protein) such as an antibody light chain.

The nucleotide sequence of CMV IE enhancer can be as shown herein (see SEQ ID NO: 1, for example). The term CMV IE enhancer includes derivatives having at least 100 consecutive nucleotides, or at least 200 consecutive nucleotides, or at least 300 consecutive nucleotides of the CMV IE enhancer shown herein (e.g., see SEQ ID NO: 1). In these or other embodiments, the CMV IE enhancer may have from 1 to 50, or from 1 to 25, or from 1 to 10) nucleotide modifications independently selected from nucleotide substitutions, deletions, and insertions, without impacting the ability of the enhancer to support expression from the expression cassette. In various embodiments, the cis-acting elements are retained and unmodified. Meier J L. Et al., Requirement of Multiple cis-Acting Elements in the Human Cytomegalovirus Major Immediate-Early Distal Enhancer for Viral Gene Expression and Replication. J. Virology Vol. 76. Issue 1 (2002). In some embodiments, the CMV IE enhancer has the nucleotide sequence of this element shown in SEQ ID NO: 1.

The nucleotide sequence of chicken beta-actin promoter (CBA) can be as shown herein (see SEQ ID NO: 1, for example). The term chicken beta-actin promoter (CBA) includes derivatives having at least 100 consecutive nucleotides, or at least 200 consecutive nucleotides, or at least 250) consecutive nucleotides of the CBA promoter shown herein (e.g., see SEQ ID NO: 1). In these or other embodiments, the CBA promoter may have from 1 to 50, or from 1 to 25, or from 1 to 10 nucleotide modifications independently selected from nucleotide substitutions, deletions, and insertions, without impacting the ability of the promoter to support expression from the expression cassette. In various embodiments, the cis-acting elements are retained and unmodified. Seo H W. Evaluation of combinatorial cis-regulatory elements for stable gene expression in chicken cells. BMC Biotechnol. 2010; 10: 69. In some embodiments, the CBA promoter has the nucleotide sequence of this element shown in SEQ ID NO: 1.

The nucleotide sequence of CMV IE intron A can be as described herein (see SEQ ID NOS: 1 or 3, for example). The term CMV IE intron A includes derivatives having at least 100 consecutive nucleotides, or at least 200 consecutive nucleotides, or at least 300 consecutive nucleotides of the CMV IE intron A contained in SEQ ID NO: 1 or SEQ ID NO: 3. In these or other embodiments, the CMV IE intron A may have from 1 to 50, or from 1 to 25, or from 1 to 10 nucleotide modifications independently selected from nucleotide substitutions, deletions. and insertions, without impacting the ability of the intron to support expression from the expression cassette. In various embodiments, the cis-acting elements are retained and are unmodified. Chapman B S, et al., Effect of intron A from human cytomegalovirus (Towne) immediate-early gene on heterologous expression in mammalian cells. Nucleic Acids Res. 1991 Jul. 25: 19 (14): 3979-3986. In some embodiments, the CMV IE intron A has the nucleotide sequence of this element shown in SEQ ID NO: 1 or SEQ ID NO: 3.

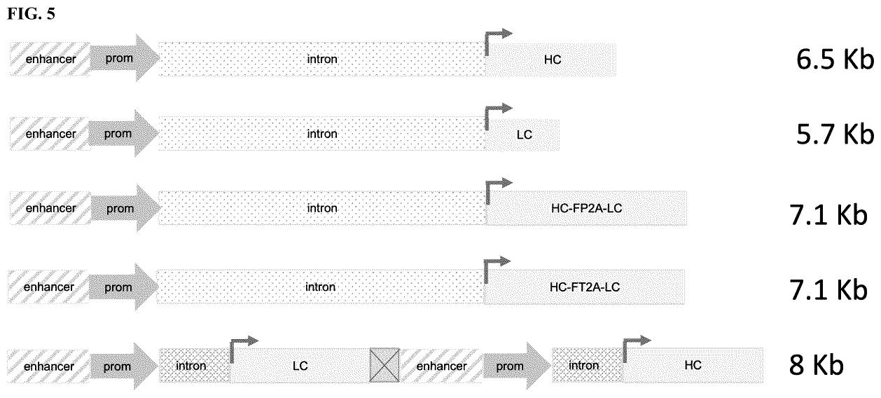

In some embodiments, the open reading frame encodes an antibody. For example, antibody heavy and light chains can be encoded by separate expression cassettes contained by the plasmid in tandem, or encoded by a single expression cassette as described herein. (see e.g., FIG. 5). In some embodiments, the open reading frame encodes a single chain antibody, such as a single chain variable fragment (scFv). In some embodiments, the antibody has an isotype of IgG, IgA, IgD, IgE, or IgM. Any subtype can be employed, including IgG1, IgG2, IgG3, and IgG4.

In some embodiments, the antibody is a mammalian antibody. For example, in some embodiments, the mammalian antibody is selected from a human antibody, a porcine antibody, a mouse antibody, a rabbit antibody, a goat antibody, a horse antibody, a chicken antibody, a hamster antibody, a sheep antibody, a monkey antibody, and a camelid antibody (e.g., a single-variable, heavy chain-only antibody antibody).

In some embodiments, the antibody, is a humanized antibody. Humanized antibodies may be chimeric immunoglobulins, immunoglobulin chains or fragments thereof (such as Fv, Fab, Fab′, F(ab′)2 or other antigen-binding subsequences of antibodies) that contain minimal sequence derived from non-human immunoglobulin. For the most part, humanized antibodies are human immunoglobulins (recipient antibody) in which residues from a complementary-determining region (CDR) of the recipient are replaced by residues from a CDR of a non-human species (donor antibody) such as mouse, rat or rabbit having the desired specificity, affinity, and capacity. In general, the humanized antibody comprises substantially all of at least one, and typically two variable domains, in which all or substantially all of the CDR regions correspond to those of a non-human immunoglobulin and all or substantially all of the framework regions are those of a human immunoglobulin sequence. The humanized antibody may include at least a portion of an immunoglobulin constant region (Fc), typically that of a human immunoglobulin. Thus, in some embodiments, the antibody is a humanized antibody. For example, in some embodiments, the humanized antibody is selected from a humanized mouse, humanized porcine, a humanized rabbit, a humanized goat, a humanized horse, a humanized chicken, a humanized sheep, or a humanized monkey antibody.

In some embodiments, the antibody is chimeric. As used herein, the term “chimeric” refers to an antibody having at least some portion of at least one variable domain derived from the antibody amino acid sequence of a non-human mammal, e.g., a rodent, or a reptile, while the remaining portions of the antibody, or antigen-binding fragment thereof, are derived from a human.

In some embodiments, the therapeutic protein is a single chain antibody or antibody mimetic, including for example a single-domain antibody, a recombinant heavy-chain-only antibody (VHH), a single-chain antibody (scFv), a shark heavy-chain-only antibody (VNAR), a microprotein (cysteine knot protein, knottin), a DARPin: a Tetranectin: an Affibody: a Transbody: an Anticalin: an AdNectin: an Affilin; a Microbody: a peptide aptamer: an alterase: a plastic antibody: a phylomer: a stradobody: a maxibody: an evibody: a fynomer, an armadillo repeat protein, a Kunitz domain, an avimer, an atrimer, a probody, an immunobody, a triomab, a troybody: a pepbody: a vaccibody, a UniBody: Affimers, a DuoBody, a Fv, a Fab, a Fab′, and/or a F(ab′)2.

Other therapeutic proteins that can be expressed by the expression cassette are described elsewhere herein, and include various protein and peptide hormones, cytokines, enzymes (e.g., for enzyme replacement therapy), and other protein molecules with therapeutic utility. Proteins and peptides may be encoded as fusion proteins with albumin or IgG Fc (e.g., IgG1, IgG2, or IgG4 Fc), to provide for half-life enhancement.

In some embodiments, the termination sequence is an artificial transcription termination sequence, and may include segments of a bovine growth hormone polyadenylation signal (BGHpA), and may include multiple polyadenylation sequences in tandem. The artificial transcription termination sequence initiates the process of releasing a newly synthesized RNA. Terminator sequences are typically found directly after 3′ regulatory elements, such as the polyadenylation or poly(A) signal, which can contribute to a stable RNA transcript. In some embodiments, the artificial transcription terminator sequence promotes RNA processing and enhances gene-expression. In some embodiments, the artificial transcription terminator sequence comprises sequence from the bovine growth hormone(bGH) gene, and may include two synthetic poly(A) sequences. An exemplary nucleotide sequence for a termination sequence is shown in SEQ ID NO: 1, for example. The term BGHpA includes derivatives having at least 100 consecutive nucleotides, or at least 200 consecutive nucleotides, or at least 250) consecutive nucleotides of the termination sequence shown herein (e.g., see SEQ ID NO: 1). In these or other embodiments, the BGHpA termination sequence may have from 1 to 50, or from 1 to 25, or from 1 to 10 nucleotide modifications independently selected from substitutions, deletions, and insertions, without impacting the ability of the termination sequence to support transcription termination and processing of expressed transcripts. In various embodiments, the cis-acting elements are retained and are unmodified. In some embodiments, the BGHpA has the nucleotide sequence of this element as shown in SEQ ID NO: 1.

In some embodiments, the expression cassette encodes two polypeptides. Polypeptides can be encoded by cDNA, or may include introns. The polypeptides can be two different therapeutic polypeptides, or can be antibody heavy and light chains. In these embodiments, the polypeptides can be encoded with a peptide linker therebetween that induces ribosomal skipping, thereby allowing for both polypeptides to be produced without translational fusion. Ribosomal skipping is a translational process wherein a viral peptide disrupts or prevents the ribosome from covalently linking (e.g., via inhibition of a peptidyl transferase) a new amino acid during translation, thereby cleaving the nascent protein and allowing translation to proceed. In some embodiments, the result of ribosomal skipping is co-translational cleavage of the nascent polyprotein. Exemplary such peptides include P2A peptide (e.g., from porcine teschovirus 1) and T2A peptide (e.g., asigna virus capsid protein). Nucleotide sequences encoding these peptides are shown in SEQ ID NOS: 5 and 8. Amino acid sequences for such peptides (and exemplary linkers) are shown in the constructs of SEQ ID NOS: 17, 18, 21, and 22.

To remove remaining amino acids from the upstream polypeptide (remaining from the peptide inducing ribosomal skipping), a proteolytic cleavage site can be incorporated on the N-terminal side of the peptide that induces ribosomal skipping. In some embodiments, the proteolytic cleavage site is a furin cleavage site, which can have the consensus sequence RXR/K-R. In some embodiments, the furin recognition site can be incorporated on the N-terminal side of the peptide that induces ribosomal skipping, optionally with a linker peptide therebetween, such as a linker of 2 to about 20 amino acids, or a linker of from about 2 to about 10 amino acids. While any linker can be used, in some embodiments flexible linkers such as linkers composed of Gly and Ser are preferred. An exemplary linker is Gly Ser Gly. In some embodiments, linkers can be selected from flexible and rigid peptide linkers. In some embodiments, flexible linkers are predominately or entirely composed of small and/or polar residues such as Gly, Ser, and Thr. An exemplary flexible linker comprises (GlyₓSer)ₙ linkers, where x is from 1 to 10 (e.g., from 2 to 6), and n is from 1 to about 10, and in some embodiments, is from 2 to about 6. In exemplary embodiments, x is from 2 to 4, and n is from 2 to 4. Due to their flexibility, these linkers are substantially unstructured. More rigid linkers include polyproline or poly Pro-Ala motifs and a-helical linkers. Generally, linkers of varying rigidity can be predominately composed of amino acids selected from Gly, Ser, Thr, Ala, and Pro. Exemplary linker sequences contain at least 5 amino acids, and may be in the range of 5 to 30 amino acids or in the range of 5 to 20 amino acids.

### Exemplary DNA Constructs and Delivery

In some embodiments, an exemplary construct comprises the expression cassette of SEQ ID NO: 1. In some embodiments, SEQ ID NO: 1 comprises the following elements: (a) a CMV IE enhancer sequence: (b) a CBA promoter sequence: (c) a CMV IE intron A sequence; and (d) a BGHpA sequence. Therapeutic polypeptide open reading frames can be cloned into the cassette of SEQ ID NO: 1 at a cloning site downstream of the intron sequence, such that the elements are functional to direct expression of the therapeutic polypeptide. Together, such elements provide for high expression of a therapeutic protein in mammalian cells (e.g., muscle cells). In some embodiments, the plasmid DNA construct (without the therapeutic protein open reading frame) comprises a nucleotide sequence that has at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% sequence identity with SEQ ID NO: 1, or has 100% sequence identity to SEQ ID NO: 1. In some embodiments, an exemplary cloning site of SEQ ID NO: 1 includes an open reading frame encoding an antibody heavy chain, and comprises the nucleotide sequence of SEQ ID NO: 2:

(SEQ ID NO: 2)
TCTTTTCTGCAGTCACCGTCGCTAGCCTCGA
GAGATCACTTCTGGCTAATAA.

In some embodiments, an exemplary construct comprises the expression cassette of SEQ ID NO: 3. SEQ ID NO: 3 has elements similar to SEQ ID NO: 1, but includes a shorter CMV IE intron A, and is therefore desirable for constructing dual expression cassettes, where two (or more) expression cassettes are included on the plasmid (e.g., in tandem). Dual expression cassettes may be used to express antibody heavy and light chains on a single plasmid. In some embodiments, the plasmid DNA construct comprises a nucleotide sequence that has at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or at least 99% sequence identity with SEQ ID NO: 3, or has 100% sequence identity to SEQ ID NO: 3. In some embodiments, an exemplary cloning site of SEQ ID NO: 4 (and encoding an antibody sequence) comprises the nucleotide sequence of SEQ ID NO: 4:

(SEQ ID NO: 4)
TTTTTTTTGCAGTCACCGTCGCTAGCCTCGA
GAGATCACTTCTGGCTAATAA.

In some embodiments, an exemplary construct comprises the expression cassette of SEQ ID NO: 5. SEQ ID NO: 5 comprises the following elements: (a) an CMV IE enhancer sequence: (b) a CBA promoter sequence: (c) a CMV IE intron A sequence: (d) a furin-T2A sequence, and (e) a BGHpA sequence, which together provide for high expression of two therapeutic proteins in mammalian cells (e.g., muscle cells) (with each polypeptide coding sequence cloned in frame on either side of the furin-T2A sequence). In some embodiments, the two polypeptides are an antibody heavy chain and an antibody light chain, for co-expression to produce a desired monoclonal antibody. In some embodiments, the plasmid DNA construct comprises a nucleotide sequence having at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity, or 100% sequence identity, to SEQ ID NO: 5.

In some embodiments, an exemplary cloning site of SEQ ID NO: 5 (comprising a nucleotide sequence encoding an antibody heavy chain for expression) comprises SEQ ID NO: 6: TCTTTTCTGCAGTCACCGTCGCTAGCAGGAGAAAGAGAGGATCCAG. In some embodiments, an exemplary cloning site of SEQ ID NO: 5 (and comprising a nucleotide sequence encoding an antibody comprises chain) SEQ ID NO: 7:

GGAAGAGAACCCCGGACCTCTCGAGAGATCA
CTTCTGGCTAATAA.

In some embodiments, an exemplary construct comprises the expression cassette of SEQ ID NO: 8. SEQ ID NO: 8 comprises the following elements: (a) an CMV IE enhancer sequence: (b) a CBA promoter sequence: (c) a CMV IE intron A sequence: (d) a furin-P2A sequence, and (e) a BGHpA sequence, which together provide for high expression of two therapeutic proteins in mammalian cells (e.g., muscle cells) (with each polypeptide coding sequence cloned in frame on either side of the furin-P2A sequence). In some embodiments, the plasmid DNA construct comprises a nucleotide sequence that has at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity, or 100% sequence identity, to SEQ ID NO: 8. In some embodiments, the plasmid DNA construct (containing a nucleotide sequence encoding an antibody chain cloned in frame) comprises the nucleotide sequence of

SEQ ID NO: 9:
TCTTTTCTGCAGTCACCGTCGCTAGCAG
GAGAAAGAGAGGATCCAG.

In some embodiments, the plasmid DNA construct (containing a nucleotide sequence encoding an antibody chain cloned in frame) comprises the nucleotide sequence of SEQ ID NO: 10: TGGAAGAGAACCCCGGACCTCTCGAGAGATCACTTCTGGCTAATA.

In some embodiments, the therapeutic protein is an antibody, involving expression of heavy and light chain sequences from a single plasmid construct, or expression of one or more single chain antibodies, as already described. Antibodies may target antigens of infectious agents such as viruses, bacteria, or parasites. For example, antibodies may target a virus antigen selected from Zika virus antigen (e.g., E protein), an influenza virus antigen (e.g., HA or NA), a beta coronavirus antigen (e.g., spike antigen, including for SARS COV), human immunodeficiency virus (HIV), hepatitis virus (e.g., HCV), a herpes virus (VZV or HSV, including HSV-1 or -2), Epstein-Barr virus, CMV, or other virus disclosed elsewhere herein. In other embodiments, the antibody targets and neutralizes the action of a pro-inflammatory cytokine, such as TNF-alpha (e.g., for treatment of RA), IL-1, IL-4, IL-6 (e.g., for treatment of RA), IL-12, and IL-23 (e.g., for treatment of plaque psoriasis or Crohn's disease). In some embodiments, the antibody targets cancer cells, for example, may target HER2, PSA, TRP-2, EpCAM, GPC3, mesothelin (MSLN), and EGFR.

In various embodiments, the antibody encoded by the DNA construct is selected from Simponi (golimumab), Remicade (Infliximab), Actemra (Tocilizumab), Humira (Adalimumab), Kevzara (Sarilumab), Cimzia (Certolizumab), Rituxan (Rituximab), Stelara (Ustekinumab), Tremfya (Guselkumab), Tildrakizumab, Taltz (Ixekizumab), Cosentyx (Secukinumab), Siliq (Brodalumab), Herceptin (Trastuzumab), Brentuximab, Alemtuzumab, Arzerra (Ofatumumab), Atezolizumab, Avastin (Bevacizumab), Avelumab, Besponsa (Inotuzumab), Bexxar (Tositumomab), Blinatumomab, Blincyto (Blinatumomab), BMS 936559, Campath (Alemtuzumab), Cetuximab, Cinqair (Reslizumab), Cyramza (Ramucirumab), Daratumumab, Darzalex (Daratumumab), Denosumab, Dinutuximab, Durvalumab, Elotuzumab, Empliciti (Elotuzumab), Gazyva (Obinutuzumab), Gemtuzumab, Ibritumomab, Inotuzumab, Ipilimumab, KEYTRUDA (Pembrolizumab), Lartruvo (Olaratumab), MK-3475, MPDL3280A, Necitumumab, Nivolumab, Obinutuzumab, Ofatumumab, Olaratumab, OPDIVO (Nivolumab), Panitumumab, Perjeta (Pertuzumab), Pertuzumab, Pidilizumab, Portrazza (Necitumumab), Ramucirumab, Sylvant (Siltuximab), SKYRIZI (Risankizumab), and Ibritumomab.

Exemplary constructs are provided herein for expression of guselkumab heavy and light chains. Constructs expressing guselkumab are useful for treating, for example, plaque psoriasis. SEQ ID NO: 11 shows expression cassettes expressing guselkumab heavy chain, while SEQ ID NO: 12 shows expression cassettes expressing guslkumab light chain. SEQ ID NO: 13 shows an expression cassette expressing both the heavy and light chains, with T2A peptide providing co-translational cleavage: while SEQ ID NO: 14 shows an expression cassette expressing both the heavy and light chains, with P2A peptide providing co-translational cleavage.

Exemplary constructs are provided herein for expression of ustekinumab heavy and light chains. Constructs expressing ustekinumab are useful for treating, for example, Crohn's disease. SEQ ID NO: 15 shows expression cassettes expressing ustekinumab heavy chain, while SEQ ID NO: 16 shows expression cassettes expressing ustekinumab light chain. SEQ ID NO: 17 shows an expression cassette expressing both the heavy and light chains, with T2A peptide providing co-translational cleavage: while SEQ ID NO: 18 shows an expression cassette expressing both the heavy and light chains, with P2A peptide providing co-translational cleavage.

Exemplary constructs are provided herein for expression of risankizumab heavy and light chains. Constructs expressing risankizumab are useful for treating, for example, plaque psoriasis. SEQ ID NO: 19 shows expression cassettes expressing risankizumab heavy chain, while SEQ ID NO: 20 shows expression cassettes expressing risankizumab light chain. SEQ ID NO: 21 shows an expression cassette expressing both the heavy and light chains, with T2A peptide providing co-translational cleavage: while SEQ ID NO: 22 shows an expression cassette expressing both the heavy and light chains, with P2A peptide providing co-translational cleavage.

Other therapeutic proteins include cytokines, growth factors, protein and peptide hormones, as well enzymes and vaccine antigens (e.g., infectious disease or cancer antigens). According to any embodiments disclosed herein, proteins encoded by the construct may contain detectable tags (such as antigen tags), to allow expression to be monitored over time.

In some embodiments, the open reading frame encodes a granulocyte colony stimulating factor (G-CSF) (e.g., filgrastim). An exemplary human G-CSF amino acid sequence that may be encoded is provided herein as SEQ ID NOS: 23. As shown herein, G-CSF may contain capturable tags such as HIS, HiBiT, and NLuc, as illustrated by SEQ ID NOS: 23 to 27. Constructs expressing G-CSF find use for, among other things, treatment of chronic neutropenia.

In some embodiments, the open reading frame encodes erythropoietic (EPO) (e.g., epoetin alfa), insulin (e.g., single chain insulin), growth hormone, a cytokine, or an insulin-like growth factor. For example, the open reading frame may encode an interferon (e.g., IFNα or IFNγ), Interleukin 2 (IL-2), Interleukin 15 (IL-15), Interleukin 12 (IL-12), or Interleukin 10 (IL-10). In some embodiments, the therapeutic protein is a glucagon-like peptide 1 (GLP-1) receptor agonist, GIP receptor agonist, or glucagon receptor agonist. In some embodiments, the therapeutic peptide is a dual or triple agonist at GLP-1, GIP, and GCG receptors. Such agonists are well known in the art, and may be used for the treatment of metabolic diseases, including diabetes mellitus and obesity. In some embodiments, such peptides are encoded as an Fc fusion to provide for half-life enhancement.

In some embodiments, the therapeutic protein is an enzyme, e.g., for enzyme replacement therapy, which may be useful for the treatment of numerous inheritable disorders. Exemplary such enzymes are disclosed elsewhere herein.

Plasmid constructs can be formulated for administration, for example, by intramuscular injection. Pharmaceutical compositions include, without limitation, solutions, emulsions, aqueous suspensions, and liposome-containing formulations. Compositions and formulations can contain sterile aqueous solutions, which also can contain buffers, diluents, and other suitable additives (e.g., penetration enhancers, carrier compounds and other pharmaceutically acceptable carriers).

In various embodiments, constructs are administered by intramuscular injection, and further comprising electroporation to facilitate entry of the plasmid into muscle cells. An exemplary device and process for intramuscular injection is disclosed in U.S. provisional application No. 63/338,774, which is hereby incorporated by reference in its entirety. For example, a device for gene transfer may comprise a pulse generator, a handpiece, and an array of electrodes arranged to maximize expression of a plasmid DNA construct delivered therethrough (through an injection needle tip contained within the device) while minimizing applied voltage and total electrical dose. For example, in some embodiments, the electrode array comprises 6 electrodes and a single DNA injection port. In some embodiments, the electrodes are arranged in a hexagonal pattern, which can provide for more consistent delivery into muscle cells. In some embodiments, the pulse(s) comprise perpendicular pulses and/or parallel pulses relative to the orientation of a muscle fiber.

### Methods of Treating or Preventing Diseases

In some embodiments, the compositions of the disclosure find use in the treatment or prevention of various diseases or disorders. In some embodiments, the methods disclosed herein prevent an onset or progression of a disease, such as an infectious disease, inflammatory disease, metabolic disease, inheritable disease (e.g., resulting in enzyme deficiency), or cancer. Various diseases and conditions and therapeutic agents can be as already described, and as further described below. In this aspect, the method comprises administering a DNA composition of the disclosure to a patient. In various embodiments, the constructs and methods described herein provide for stable expression (e.g., in muscle cells) for at least about 4 months, at least about 6 months, at least about 1 year, or at least about 18 months, or at least about 2 years. In some embodiments, e.g., for the treatment of an infectious disease such as a virus, the subject receives a single administration of the plasmid construct. In other embodiments, particularly for chronic disease, the subject may receive repeated dosing, which may be no more frequent than quarterly, or twice per year, or once per year, or about once every two years, in various embodiments. Subjects are generally mammalian subjects, such as human subjects, but in other embodiments may be veterinary subjects (e.g., dog, cat, horse, or pig) or livestock (e.g., cow, pig, sheep, etc.).

As disclosed herein, administering, or administering a treatment/therapy, refers to a treatment/therapy from which a subject receives a beneficial effect, such as the reduction, decrease, attenuation, diminishment, stabilization, remission, suppression, inhibition or arrest of the development or progression of cancer, or a symptom thereof.

In various embodiments, disclosed herein is a method for treating or preventing (e.g., in a subject at risk of) a viral infection, comprising administering an effective amount of the composition to a patient in need thereof. In some embodiments, administering includes at least one of electroporation and injection. In some embodiments, the administering is intramuscular injection. In some embodiments, the administering comprises applying a stimulus to a muscle cell in the patient. In some embodiments, the stimulus is an electrical pulse. In some embodiments, the antibody or the therapeutic protein is expressed in the muscle cell. In some embodiments, the therapeutic protein is an antibody (as already described) that neutralizes the virus, many of which are known in the art. In some embodiments, the method further comprises detecting and/or monitoring the antibody or the therapeutic protein in the patient's blood.

In some embodiments, the virus is of the family Arbovirus, Arenaviridae, Arterivirus, Astroviridae, Bimaviridae, Bromo viridae, Bunyaviridae, Caliciviridae, Circoviridae, Closteroviridae, Comoviridae, Coronaviridae, Cystoviridae, Filoviridae, Flaviviridae, Flexiviridae, Hepadnaviridae, Hepevirus, Herpesviridae, Leviviridae, Luteoviridae, Mesoniviridae, Mononegavirales, Mosaic Viruses, Nidovirales, Nodaviridae, Orthomyxoviridae, Papillomaviridae, Papovaviridae, Parvoviridae, Paramyxoviridae, Picobirnaviridae, Picobimavirus, Picornaviridae, Poty viridae, Poxviridae, Reoviridae, Retroviridae, Roniviridae, Sequiviridae, Tenuivirus, Togaviridae, Tombusviridae, Totiviridae, or Tymoviridae. In some embodiments, the viral infection is selected from Alfuy virus, Banzi virus, bovine diarrhea virus, Chikungunya virus. Dengue virus (DNV). Epstein Barr Virus (EBV). Hepatitis B virus (HBV), Hepatitis C virus (HCV), herpes simplex virus type 1 (HSV-1), herpes simplex virus type 2 (HSV-2), human cytomegalovirus (hCMV), human immunodeficiency virus (HIV), Ilheus virus, influenza virus (including avian and swine isolates), rhinovirus, norovirus, adenovirus, Japanese encephalitis virus, Kaposi's sarcoma associated herpesvirus (KSHV), Kokobera virus, Kunjin virus, Kyasanur forest disease virus, louping-ill virus, measles virus, MERS-coronavirus (MERS), metapneumovirus, any of the Mosaic Viruses, Murray Valley virus, parainfluenza virus, poliovirus, Powassan virus, respiratory syncytial virus (RSV), Rocio virus, SARS-coronavirus (SARS), St. Louis encephalitis virus, tick-home encephalitis vims, West Nile virus (WNV), Ebola virus, Nipah virus, Lassa vims, Tacaribe virus, Junin vims, yellow fever vims, Varicella zoster virus (VZV) (e.g., for treatment or prevention of shingles), and vesicular stomatitis virus. In such embodiments, the therapeutic agent (e.g., an antibody) binds to and neutralizes virus in the subject. In some embodiments, the subject has or is at risk of zika virus.

In various embodiments, disclosed herein is a method for treating or preventing an inflammatory or autoimmune disease or disorder, comprising administering an effective amount of the composition to a patient in need thereof. In some embodiments, administering includes at least one of electroporation and injection. In some embodiments, the administering is intramuscular injection. In some embodiments, the administering comprises applying a stimulus to a muscle cell in the patient. In some embodiments, the stimulus is an electrical pulse. In some embodiments, the antibody or the therapeutic protein is expressed in the muscle cell. In some embodiments, the method further comprises detecting or monitoring the antibody or the therapeutic protein in the patient's blood. In some embodiments, the inflammatory or autoimmune disease or disorder is selected from graft versus host disease, transplantation rejection (e.g., prevention of allograft rejection), multiple sclerosis, diabetes mellitus, celiac disease, Crohn's disease, pediatric Crohn's disease, ulcerative colitis, Guillain-Barre syndrome, scleroderma, Goodpasture's syndrome, Wegener's granulomatosis, autoimmune epilepsy, Rasmussen's encephalitis, Primary biliary sclerosis, Sclerosing cholangitis, Autoimmune hepatitis, Addison's disease, Hashimoto's thyroiditis, Fibromyalgia, Meniere's syndrome: pernicious anemia, rheumatoid arthritis, systemic lupus erythematosus, dermatomyositis, Sjogren's syndrome, lupus erythematosus, multiple sclerosis, myasthenia gravis, Reiter's syndrome, Grave's disease, rheumatoid arthritis, psoriatic arthritis, plaque psoriasis, ankylosing spondylitis, and juvenile idiopathic arthritis. In some embodiments, the autoimmune disease or disorder is graft versus host disease. In some embodiments, the inflammatory disease is plaque psoriasis or Crohn's disease. Exemplary constructs expressing risankizumab, guselkumab, and ustekinumab are disclosed herein, which are useful for such embodiments.

In various embodiments, disclosed herein is a method for treating or preventing cancer, comprising administering an effective amount of the DNA composition of the present disclosure to a patient in need thereof. For example, in certain embodiments, the construct may express an antibody or protein that targets HER2, PSA, TRP-2, EpCAM, GPC3, mesothelin (MSLN), or EGFR. In some embodiments, the DNA construct expresses a bispecific T cell engager (BiTE). Various BiTE constructs (including anti-CD3 and anti-CD19) are known in the art.

In some embodiments, the subject has cancer, which is optionally stage 1, stage 2, stage 3, or stage 4. In some embodiments, the subject has stage 1 or stage 2 cancer, and the DNA construct is administered following tumor resection. In various embodiments, the DNA construct is administered with or following chemotherapy, radiation therapy, or cell therapy that is optionally a T cell therapy (e.g., CAR-T therapy).

In some embodiments, the administering includes at least one of electroporation and injection. In some embodiments, the administering is intramuscular injection. In some embodiments, the administering comprises applying a stimulus to a muscle cell in the patient. In some embodiments, the stimulus is an electrical pulse. In some embodiments, the antibody or the therapeutic protein is expressed in the muscle cell. In some embodiments, the method further comprises detecting or monitoring the antibody or the therapeutic protein in the patient's blood. In some embodiments, the cancer is a solid tumor. In some embodiments, the cancer is a blood cancer. In some embodiments, the cancer is selected form one or more of a cancer of a blood vessel, an eye tumor, basal cell carcinoma, biliary tract cancer; bladder cancer; bone cancer; brain and central nervous system cancer; primary breast cancer; metastatic breast cancer, colorectal cancer, cancer of the peritoneum; cervical cancer; choriocarcinoma: colon and rectum cancer; connective tissue cancer; cancer of the digestive system: endometrial cancer; esophageal cancer; eye cancer; cancer of the head and neck; gastric cancer (including gastrointestinal cancer); glioblastoma; hepatic carcinoma; hepatoma; intra-epithelial neoplasm: kidney or renal cancer; larynx cancer; leukemia: liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma (e.g., Kaposi's sarcoma); skin cancer; squamous cell cancer; stomach cancer; testicular cancer; thyroid cancer; uterine or endometrial cancer; cancer of the urinary system: vulvar cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g. that associated with brain tumors), and Meigs' syndrome.

In various embodiments, disclosed herein is a method for treating or preventing an inflammatory eye disease, comprising administering an effective amount of the composition of any one of the preceding embodiments to a patient in need thereof. In some embodiments, administering includes at least one of electroporation and injection. In some embodiments, the administering is intramuscular injection. In some embodiments, the administering comprises applying a stimulus to a muscle cell in the patient. In some embodiments, the stimulus is an electrical pulse. In some embodiments, the antibody or the therapeutic protein is expressed in the muscle cell. In some embodiments, the method further comprises detecting or monitoring the antibody or the therapeutic protein in the patient's blood. In some embodiments, the administering increases the uptake of the antibody or the therapeutic protein in the patient's blood. In some embodiments, the inflammatory eye disease is selected from an inflammatory eye disease associated with corneal transplant, diabetic macular edema, diabetic retinopathy, dry eye disease, scleritis, blepharitis, keratitis, conjunctivitis, chorioretinal inflammation, chorioretinitis, iridocyclitis, iritis, posterior cyclitis, and uveitis. In some embodiments, the inflammatory eye disease is associated with a corneal allograft, or a corneal allograft rejection. In some embodiments, the uveitis is anterior uveitis, panuveitis, intermediate uveitis and posterior uveitis.

In various embodiments, disclosed herein is a method for improving a patient response to allogeneic hematopoietic stem cell transplantation (aHSCT), comprising administering an effective amount of the composition to a patient in need thereof. In some embodiments, administering includes at least one of electroporation and injection. In some embodiments, the administering is intramuscular injection. In some embodiments, the administering comprises applying a stimulus to a muscle cell in the patient. In some embodiments, the stimulus is an electrical pulse. In some embodiments, the antibody or the therapeutic protein is expressed in the muscle cell. In some embodiments, the method further comprises detecting the antibody or monitoring the therapeutic protein in the patient's blood. In some embodiments, the administering increases the uptake of the antibody or the therapeutic protein in the patient's blood.

In various embodiments, disclosed herein is a method for treating or preventing a rare disease (e.g., an inheritable disease), comprising administering an effective amount of the composition to a patient in need thereof. In some embodiments, administering includes at least one of electroporation and injection. In some embodiments, the administering is intramuscular injection. In some embodiments, the administering comprises applying a stimulus to a muscle cell in the patient. In some embodiments, the stimulus is an electrical pulse. In some embodiments, the antibody or the therapeutic protein is expressed in the muscle cell. In some embodiments, the method further comprises detecting or monitoring the antibody or the therapeutic protein in the patient's blood. In some embodiments, the rare disease is selected from severe chronic neutropenia, WHIM Syndrome, Aminoacylase 1 deficiency. Apo A-I deficiency, Carbamoyl phosphate synthetase 1 deficiency, Omithine transcarbamylase deficiency, Plasminogen activator inhibitor type 1 deficiency, Flaujeac factor deficiency, High-molecular-weight kininogen deficiency congenital, PEPCK 1 deficiency, Pyruvate kinase deficiency liver type, Alpha 1-antitrypsin deficiency, Anti-plasmin deficiency congenital, Apolipoprotein C 2I deficiency, Butyrylcholinesterase deficiency, Complement component 2 deficiency, Complement component 8 deficiency type 2, Congenital antithrombin deficiency type 1, Congenital antithrombin deficiency type 2, Congenital antithrombin deficiency type 3, Cortisone reductase deficiency 1, Factor VII deficiency, Factor X deficiency, Factor XI deficiency, Factor XII deficiency, Factor XIII deficiency, Fibrinogen deficiency congenital, Fructose-1 6-bisphosphatase deficiency, Gamma aminobutyric acid transaminase deficiency, Gamma-cystathionase deficiency, Glut2 deficiency, GTP cyclohydrolase I deficiency, Isolated growth hormone deficiency type 1B, Molybdenum cofactor deficiency, Prekallikrein deficiency congenital, Proconvertin deficiency congenital, Protein S deficiency, Pseudocholinesterase deficiency, Stuart factor deficiency congenital, Tetrahydrobiopterin deficiency, Type 1 plasminogen deficiency, Urocanase deficiency, Chondrodysplasia punctata with steroid sulfatase deficiency, Homocystinuria due to CBS deficiency, Guanidinoacetate methyltransferase deficiency, Pulmonary surfactant protein B deficiency, Acid Sphingomyelinase Deficiency, Adenylosuccinate Lyase Deficiency, Aggressive Angiomyxoma, Albrights Hereditary Osteodystrophy, Carney Stratakis Syndrome, Carney Triad Syndrome, CDKL5 Mutation, CLOVES Syndrome, Cockayne Syndrome, Congenital Disorder of Glycosylation type 1R, Cowden Syndrome, DEND Syndrome, Dercum's Disease, Febrile Infection-Related Epilepsy Syndrome, Fibular Aplasia Tibial Campomelia Oligosyndactyly Syndrome, Food Protein-Induced Enterocolitis Syndrome, Foreign Body Giant Cell Reactive Tissue Disease, Galloway-Mowat, Gitelman syndrome, Glycerol Kinase Deficiency, Glycogen Storage Disease type 9, gml gangliosidosis, Hereditary spherocytosis, Hidradenitis Suppurativa Stage III, Horizonatal Gaze Palsy with Progressive Scoliosis, IMAGe syndrome, Isodicentric chromosome 15, isolated hemihyperplasia, Juvenile Xanthogranuloma, Kasabach-Merritt Syndrome, Kniest Dysplasia, Koolen de-Vries Syndrome, Lennox-Gastaut syndrome, Lymphangiomatosis, Lymphangiomiomytosis, MASA Syndrome, Mast Cell Activation disorder, Mecp2 Duplication Syndrome, Mucha Habermann, Neonatal Hemochromatosis, N-glycanase deficiency, Opsoclonus Myoclonus Syndrome, Persistent genital arousal disorder, Pompe Disease, Progressive Familial Intrahepatic Cholestasis, Pseudohypoparathyroidism type 1a, PTEN Hamartoma Tumor Syndrome, Schnitzler syndrome, Scleroderma, Semi Lobar Holoprosencephany, Sjogren's Syndrome, Specific Antibody Deficiency Disease, SYNGAP 1 deficiency, Trigeminal Trophic Syndrome, Undifferentiated Connective Tissue Disease, or X-linked hypophosphatemia. In various embodiments, the subject suffers from an enzyme deficiency, which is treated according to this disclosure by delivering a construct expressing such enzyme for enzyme replacement therapy.

In some aspects and embodiments, the disclosure provides a method for treating a metabolic disease, diabetes mellitus, or obesity. In such embodiments, the therapeutic protein is a glucagon-like peptide 1 (GLP-1) receptor agonist, GIP receptor agonist, or glucagon (GCG) receptor agonist. In some embodiments, the therapeutic peptide is a dual or triple agonist at GLP-1, GIP, and GCG receptors. In some embodiments, such peptides are encoded as an Fc fusion to provide for half-life enhancement.

As used herein, unless the context requires otherwise, the term about means±10% of an associated numerical value.

As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features. Although the open-ended term “comprising,” as a synonym of terms such as including, containing, or having, is used herein to describe and claim the disclosure, the present technology, or embodiments thereof, may alternatively be described using more limiting terms such as “consisting of” or “consisting essentially of” the recited ingredients.

Unless defined otherwise, all technical and scientific terms herein have the same meaning as commonly understood by one of ordinary skill in the art to which this disclosure belongs. Although any methods and materials, similar or equivalent to those described herein, can be used in the practice or testing of the present disclosure, the preferred methods and materials are described herein. All publications, patents, and patent publications cited are incorporated by reference herein in their entirety for all purposes.

This disclosure is further illustrated by the following non-limiting examples.

### EXAMPLES

### Example 1: Design and Application of Plasmid DNA Constructs to Treat a Variety of Diseases

In the experiments of this example, plasmid DNA constructs were designed, as shown in FIG. 5. The plasmid DNA constructs (see the first four DNA constructs in FIG. 5, from top to bottom) were designed to comprise a) an enhancer sequence (CMV IE enhancer), b) a promoter sequence (CBA promoter), c) an intron sequence (CMV IE intron A), and d) a transcription termination. The red arrow in each construct of FIG. 5 indicates the location of a cloning site where a therapeutic protein can be encoded. Antibody heavy and light chains may be expressed in a single reading frame, using furin T2A or furin P2A to allow for co-translational cleavage.

The experiments of this example also demonstrate dual expression constructs (last DNA construct in FIG. 5, appearing at the bottom of the figure: SEQ ID NO: 3). This construct permits expression of an antibody heavy chain and an antibody light chain from a single plasmid, using two expression cassettes.

In the experiments of this example, an electroporation device for gene transfer was used to deliver the DNA constructs to the mice. As disclosed herein, the electroporation device has a handpiece, an array of electrodes arranged at one end of the handpiece and configured to be positioned at a host cell of a subject; and a pulse generator configured to generate electric pulses that cause the array of electrodes to emit electric fields in the targeted tissue to maximize expression of a plasmid DNA construct delivered therethrough while minimizing applied voltage and total electrical dose. The device for intramuscular injection is disclosed in U.S. provisional application No. 63/338,774, which is hereby incorporated by reference in its entirety.

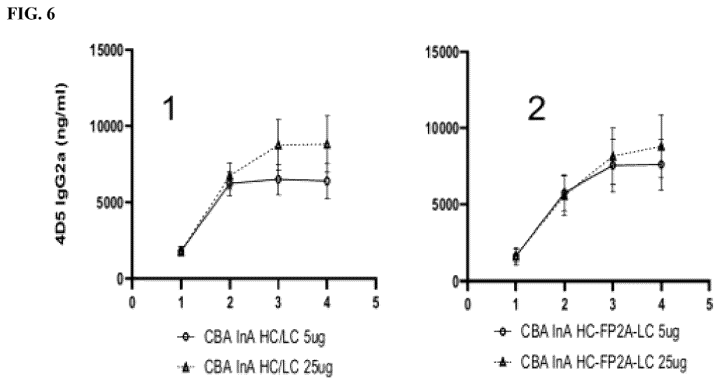

In the experiments shown in FIG. 6, mice were administered different plasmid DNA constructs encoding a murine version of the monoclonal antibody trastuzumab (4D5), and the serum antibody concentrations of 4D5 were measured at weeks 1˜4 post administration. When mice were administered a total of either 5 μg or 25 μg of two plasmids, one encoding the heavy chain (HC) and one encoding the light chain (LC) of 4D5 (panel 1), the higher DNA dose was required in order to achieve a maximum serum antibody concentration. In contrast, when animals were administered the same doses of plasmid DNA in which both the HC and LC were encoded in a single plasmid (a single expression cassette with co-translational cleavage) (panel 2), the lower dose of 5 μg was sufficient to achieve nearly the same serum antibody concentration as the higher dose of 25 μg.

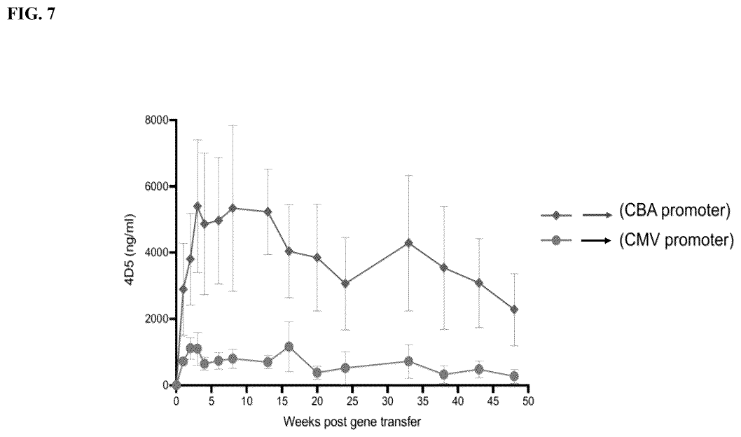

The experiments shown in FIG. 7 demonstrate testing of different promoters in the plasmid DNA constructs. Two groups of mice were administered 5 μg of a plasmid DNA construct encoding a murine version of the monoclonal antibody trastuzumab (4D5) at week 0 (i.e. intramuscular injection), and serum antibody concentrations were measured at the indicated timepoints following administration. The plasmids that both groups received were the same except for the promoter. One group of animals received a construct including the CMV promoter (gray line), and the other group of animals received the CMV promoter been replaced with a chicken beta-actin (CBA) promoter (red line). The animals that received the plasmid with CBA promoter had significantly higher (and surprising) levels of serum 4D5 antibody for a period of at least 48 weeks.

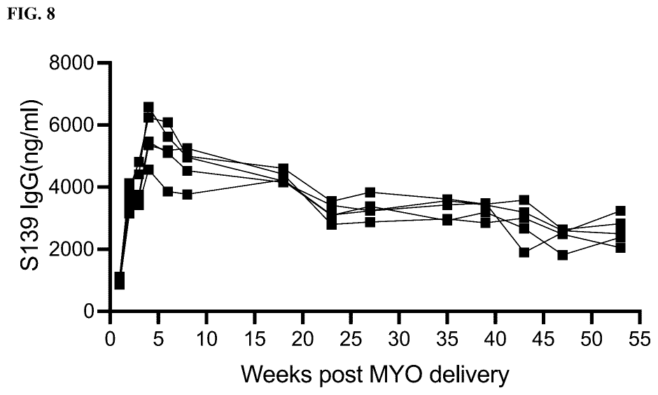

The experiments in FIG. 8 demonstrate stable antibody expression and production in mice for a period more than one year. In these experiments, mice were administered 5 μg of a plasmid DNA construct (SEQ ID NO: 8) encoding the murine antibody S139 with the electroporation device disclosed herein, and antibody levels were monitored in the mice over the time course of the experiments.

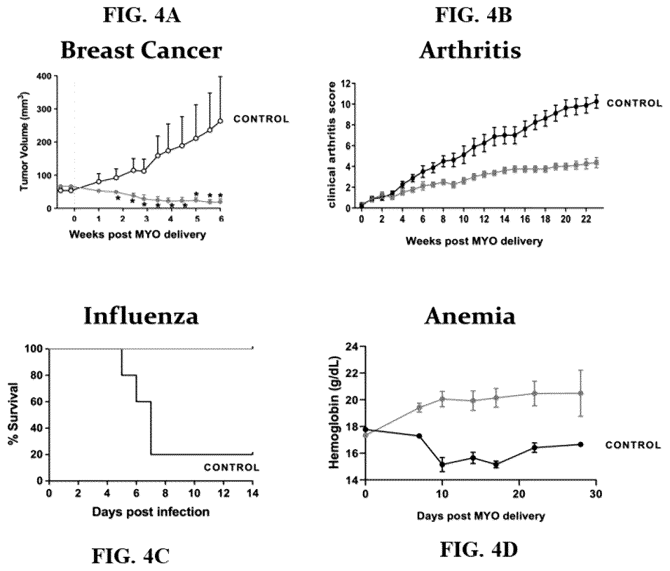

The experiments in FIG. 4A, FIG. 4B, FIG. 4C, and FIG. 4D demonstrate that plasmid DNA constructs encoding an antibody or therapeutic protein, are applicable for treating and/or preventing a variety of diseases. In FIG. 4A, mice were implanted with human breast cancer tumor cells expressing the tumor antigen HER2. At time zero (dotted line) animals were either left untreated (black line), or administered plasmid DNA encoding a murine version of the anti-HER2 monoclonal antibody, trastuzumab (Herceptin) (red line), delivered by intramuscular injection into a muscle. The delivery of trastuzumab using the plasmid DNA constructs disclosed herein significantly suppressed tumor growth, as well as decreased tumor volume, as compared to the control group.

In FIG. 4B, mice bearing a transgene for human TNF-alpha develop a progressive, inflammatory arthritis disease. These animals were either administered an empty vector plasmid DNA (black line) or a plasmid DNA construct encoding a murine version of the anti-TNF-alpha antibody certolizumab (red line), delivered by intramuscular injection into a muscle cell, at week zero and again at week 12. The y-axis shows the clinical arthritis score. Animals that received murine certolizumab based on a plasmid DNA construct had significantly slower disease progression and milder disease overall, as compared to the control group.

In FIG. 4C, mice were administered a plasmid DNA construct encoding a monoclonal antibody directed against influenza A virus (red line), delivered by intramuscular injection (as shown in FIG. 1), or left untreated (black line). At day zero, the animals were infected with a mouse-adapted H1N1 influenza A virus and monitored for virus-induced mortality. Animals that received the anti-influenza antibody based on a plasmid DNA construct were completely protected from virus-associated mortality, in contrast to the control group.

In FIG. 4D, mice were administered a plasmid DNA construct encoding the therapeutic protein erythropoietin (epoetin alfa) (red line), delivered by intramuscular injection, or left untreated (black line) at day 0. Seven days later (arrow), all the animals were treated with cisplatin to induce acute anemia. In contrast to the control animals, the animals that received erythropoietin based on a plasmid DNA construct were completely protected from anemia, as indicated by their blood hemoglobin levels.

### Example 2: Evaluation of Alternative DNA Construct Designs

In the experiments of this example, plasmid DNA constructs were evaluated for the optimal combination of the promoter, enhancer, and poly A signal to enhance the expression of a therapeutic protein in mammalian cells. While prior studies from Xu et al. (“Optimization of transcriptional regulatory elements for constructing plasmid vectors” Gene, 2001 Jul. 11; 272 (1-2): 149-56) have shown the CA part of the overall CAG promoter, as well as intron A and the BGH poly A can provide some expression in skeletal muscle cells, this promoter, enhancer, and polyA signal combination were not tested together to evaluate the expression of a therapeutic protein in mammalian cells over any duration of time. In the experiments of this example, it was hypothesized that the CBA promoter was more resistant to silencing, and thus, the combination of CBA promoter and Intron A in a DNA construct, as disclosed herein, would result in stable, high-level gene expression in skeletal muscle.

Indeed, the experiments of this example found that the combination of the CA part of the overall CAG promoter, intron A (or the Intron A deletion mutant, intron pCON3 [InpCON3]), and the BGH poly A significantly increase therapeutic protein expression level in vitro and also in skeletal muscle cells in vivo, as compared to other combinations of elements.

In the experiments of this example, the electroporation device for gene transfer was used to deliver the DNA constructs to the mice.

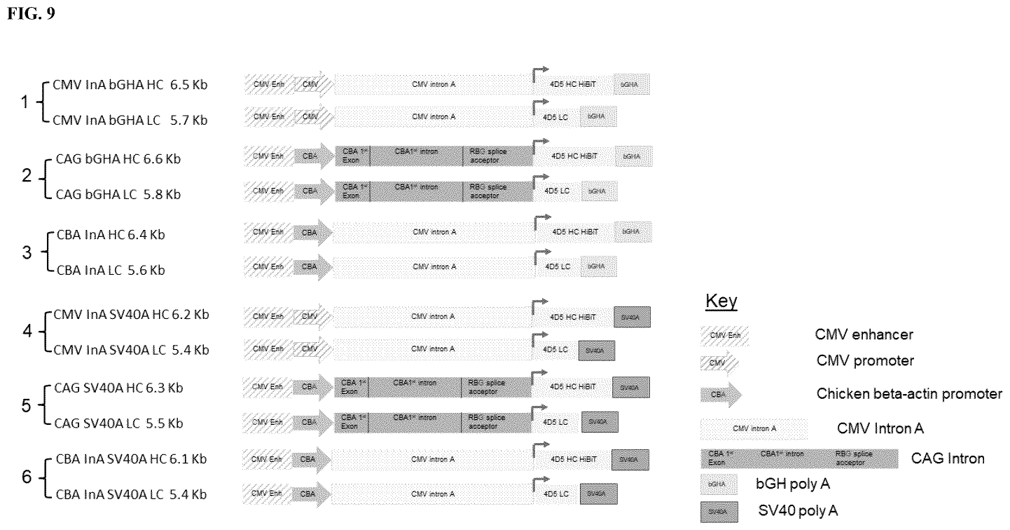

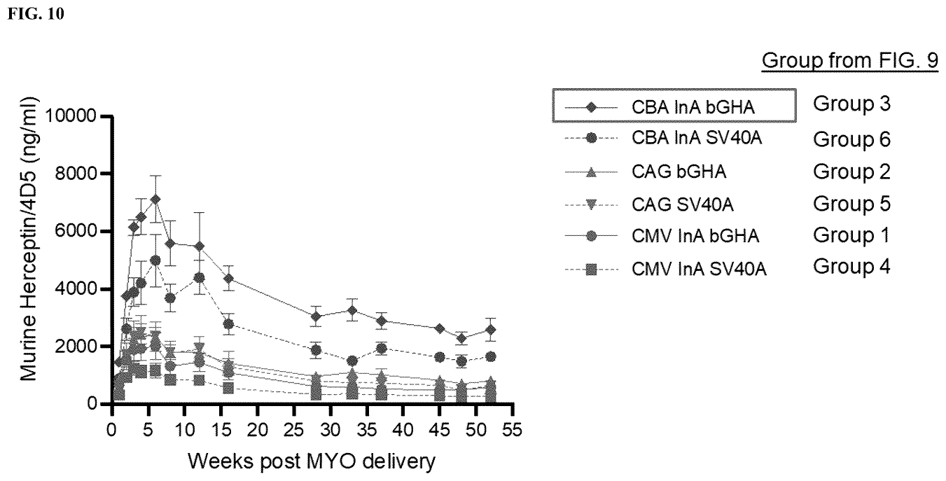

FIG. 9 shows a series of non-limiting images of DNA constructs encoding a therapeutic protein. The DNA constructs shown in FIG. 9 represent the regulatory element configurations that were used to generate the data in the experiments shown in FIG. 10. In FIG. 9, the genes of interest are the heavy and light chains of 4D5, a mouse monoclonal antibody that binds HER2 (from which Herceptin was derived), and is used herein as an illustrative example.

FIG. 10 is a graph showing the serum antibody concentration levels over a period of 1 year for different DNA constructs encoding the herceptin 4D5 antibody. In these experiments, the heavy chain and light chain antibody sequences were on two separate DNA constructs and significantly increased serum antibody concentrations were achieved with the CBA promoter, Intron A sequence, and bGHA were shown to significantly increase serum antibody concentrations.

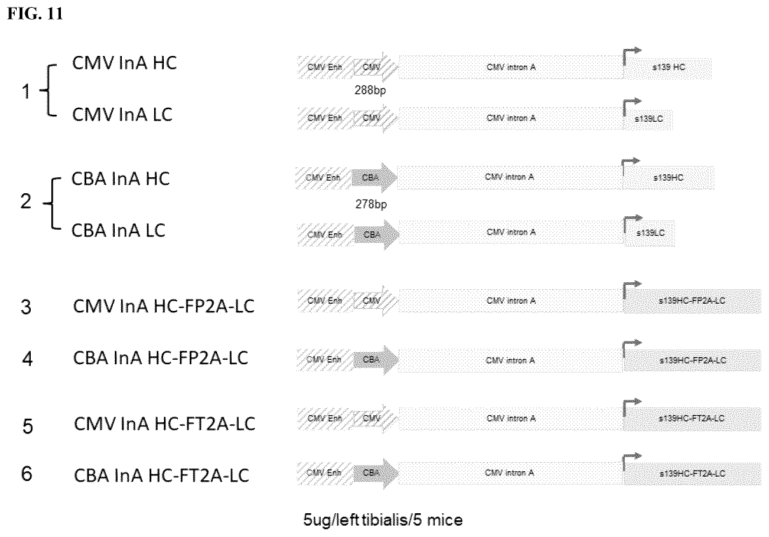

FIG. 11 shows a series of non-limiting images of DNA constructs encoding a therapeutic protein. In FIG. 11, a single cassette is one set of regulatory elements (e.g., an enhancer, promoter, intron, terminator/poly A). One advantage of a single cassette, over a dual cassette, is the ability to express a therapeutic protein, such as an antibody heavy chain and light chain, from a single plasmid by inserting a furin-2A site between the sequence encoding the antibody heavy chain and the sequence encoding light chain.

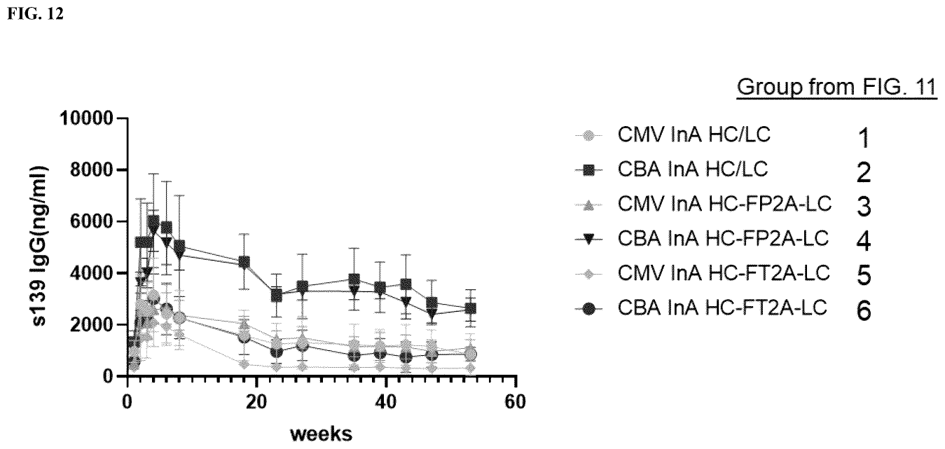

FIG. 12 is a graph showing the serum antibody concentration levels over a period of 1 year for different DNA constructs encoding a therapeutic protein (i.e., murine anti-influenza virus antibody). In these experiments, significantly increased serum antibody concentrations with the CBA promoter and Intron A sequence were shown to significantly increase serum antibody concentrations.

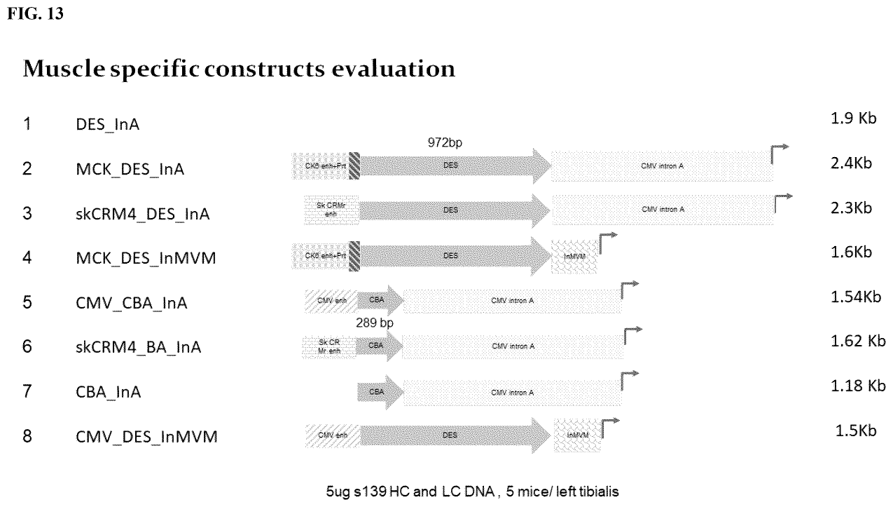

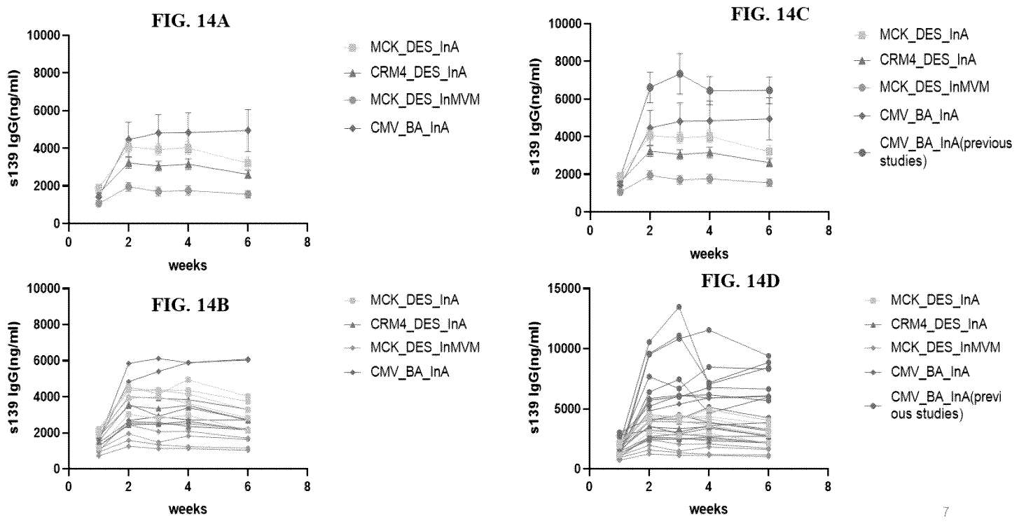

FIG. 13 shows a series of non-limiting images of DNA constructs encoding muscle specific proteins for evaluation. The DNA constructs in FIG. 14 were engineered to carry the DES (desmin) promoter, a naturally occurring promoter of the desmin gene, or the CMV promoter. The desmin protein is a muscle-specific cytoskeletal protein belonging to the intermediate filament family, and is known to have high expression levels in mammalian muscle cells. The DNA constructs in FIG. 14 were also engineered to have sequences that encode the Muscle creatine kinase (MCK) protein, which is induced to high levels during skeletal muscle differentiation.

As shown in the graphs of FIG. 14A, FIG. 14B, FIG. 14C, and FIG. 14D, the CBA InA (CMV_CBA_InA) construct (SEQ ID NO: 3) outperforms all other muscle specific promoters evaluated.

Together, these experiments demonstrate that, inter alia, the plasmid DNA constructs disclosed herein allow for the simultaneous expression and production of antibodies and therapeutic proteins in vivo at high levels, and compared to other therapeutics, will result in a significant decrease in administration frequency, and have a robust therapeutic duration. Accordingly, the plasmid DNA constructs disclosed herein allow for the treatment of multiple diseases and a variety of conditions using the patient's own cells for the expression and production of the antibody or therapeutic protein.

All of the features disclosed herein may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

What is claimed is:

1. A DNA composition comprising a plasmid construct having at least one expression cassette, wherein the expression cassette comprises in the following order from 5′ to 3′:
a) a CMV IE enhancer sequence;
b) a chicken beta-actin promoter sequence;
c) a CMV IE intron A sequence;
d) a cloning site or an open reading frame encoding a therapeutic protein; and
e) a transcription termination sequence.

2. The DNA composition of claim 1, wherein the termination sequence is an artificial transcription termination sequence comprising segments of a bovine growth hormone polyadenylation signal (BGHpA).

3. The DNA composition of claim 1 or 2, wherein the therapeutic protein is selected from an antibody, protein or peptide hormone, cytokine, or enzyme.

4. The DNA composition of claim 3, wherein the therapeutic protein is an antibody heavy and/or antibody light chain, and wherein the antibody optionally binds to and neutralizes a virus or an inflammatory cytokine, or targets a cancer cell.

5. The DNA composition of claim 4, wherein the virus is Zika virus, an influenza virus, a beta coronavirus, human immunodeficiency virus (HIV), hepatitis virus, a herpes virus, Epstein-Barr virus, or CMV.

6. The DNA composition of claim 4, wherein the antibody targets and neutralizes the action of a pro-inflammatory cytokine selected from TNF-alpha, IL-1, IL-4, IL-6, IL-12, and IL-23.

7. The DNA composition of claim 4, wherein the antibody targets a cancer antigen selected from HER2, PSA, TRP-2, EpCAM, GPC3, mesothelin (MSLN), and EGFR.

8. The DNA composition of any one of claims 3 to 7, wherein the therapeutic protein is a single chain variable fragment (scFv).

9. The DNA composition of any one of claims 3 to 7, wherein the plasmid construct comprises two of said expression cassettes, a first expression cassette having an open reading frame encoding an antibody heavy chain and a second expression cassette having an open reading frame encoding an antibody light chain.

10. The DNA composition of any one of claims 3 to 7, wherein the open reading frame encodes antibody heavy and light chains with a peptide linker therebetween that induces ribosomal skipping, and which optionally comprises a P2A peptide or a T2A peptide.

11. The DNA composition of claim 10, wherein the peptide linker comprises a P2A peptide.

12. The DNA composition of claim 10, wherein the peptide linker comprises a T2A peptide.

13. The DNA composition of any one of claims 10 to 12, wherein the peptide linker further comprises a furin recognition site on the N-terminal side of the peptide that induces ribosomal skipping.

14. The DNA composition of claim 13, wherein the linker sequence comprises a linker between the furin recognition site and the peptide that induces ribosomal skipping, wherein the Gly Ser linker is optionally Gly Ser linker.

15. The DNA composition of any one of claims 1 to 8, wherein the expression cassette comprises the nucleotide sequence of SEQ ID NO: 1, optionally with a therapeutic protein open reading frame inserted after the CMV IE intron A sequence.

16. The DNA composition of any one of claims 1 to 8, wherein the expression cassette comprises the nucleotide sequence of SEQ ID NO: 3, optionally with a therapeutic protein coding sequence inserted after the CMV IE intron A sequence.

17. The DNA composition of any one of claims 1 to 8, wherein the expression cassette comprises the nucleotide sequence of SEQ ID NO: 5, optionally with a therapeutic protein coding sequence inserted on either side of the Furin P2A sequence.

18. The DNA composition of any one of claims 1 to 8, wherein the expression cassette comprises the nucleotide sequence of SEQ ID NO: 8, optionally with a therapeutic protein coding sequence inserted on either side of the Furin T2A sequence.

19. The DNA composition of any one of claims 15 to 18, expressing a guselkumab heavy and/or light chain.

20. The DNA composition of claim 19, expressing the amino acid sequence of SEQ ID NO: 13 or SEQ ID NO: 14.

21. The DNA composition of any one of claims 15 to 18, expressing a ustekinumab heavy and/or light chain.

22. The DNA composition of claim 21, expressing the amino acid sequence of SEQ ID NO: 17 or SEQ ID NO: 18.

23. The DNA composition of any one of claims 15 to 18, expressing a risankizumab heavy and/or light chain.

24. The DNA composition of claim 23, expressing the amino acid sequence of SEQ ID NO: 21 or SEQ ID NO: 22.

25. The DNA composition of claim 3, wherein the therapeutic protein is selected from a granulocyte colony stimulating factor, an erythropoietin, and an insulin-like growth factor.

26. The DNA composition of claim 25, wherein the therapeutic protein is filgrastim.

27. The DNA composition of claim 25, wherein the therapeutic protein is epoetin alfa.

28. The DNA composition of claim 3, wherein the therapeutic protein is a GLP-1 receptor agonist, a GIP receptor agonist, and/or a glucagon receptor agonist.

29. The DNA composition of claim 28, wherein the therapeutic protein is a dual or triple agonist.

30. The DNA composition of claim 28 or 29, wherein the therapeutic protein comprises an albumin or Ig Fc fusion.

31. A method for treating or preventing a viral infection, comprising administering an effective amount of the composition of any one of claim 4 or 5 to a subject in need thereof, wherein the antibody binds to and neutralizes a virus.

32. The method of claim 31, wherein the subject has a viral infection.

33. The method of claim 31, wherein the subject is at risk of a viral infection.

34. The method of claim 31, wherein the viral infection is selected from Alfuy virus, Banzi virus, bovine diarrhea virus, Chikungunya virus, Dengue virus (DNV), Epstein Barr Virus (EBV), Hepatitis B virus (HBV), Hepatitis C virus (HCV), herpes simplex virus type 1 (HSV-1), herpes simplex virus type 2 (HSV-2), human cytomegalovirus (hCMV), human immunodeficiency virus (HIV), Ilheus virus, influenza virus (including avian and swine isolates), rhinovirus, norovirus, adenovirus, Japanese encephalitis virus, Kaposi's sarcoma associated herpesvirus (KSHV), Kokobera virus, Kunjin virus, Kyasanur forest disease virus, louping-ill virus, measles virus, MERS-coronavirus (MERS), metapneumovirus, any of the Mosaic Viruses, Murray Valley virus, parainfluenza virus, poliovirus, Powassan virus, respiratory syncytial virus (RSV), Rocio virus, SARS-coronavirus (SARS), St. Louis encephalitis virus, tick-home encephalitis vims, West Nile virus (WNV), Ebola virus, Nipah virus, Lassa vims, Tacaribe virus, Junin vims, yellow fever vims, Varicella zoster virus (VZV), and vesicular stomatitis virus.

35. The method of claim 31, wherein the virus is Zika virus, an influenza virus (A or B), a beta coronavirus, human immunodeficiency virus (HIV), hepatitis virus, a herpes virus, Epstein-Barr virus, or CMV.

36. The method of any one of claims 31 to 35, wherein the expression cassette is according to any one of claims 8 to 18.

37. The method of any one of claims 31 to 36, wherein administering comprises injection and electroporation.

38. The method of any one of claims 31 to 37, wherein the administering is intramuscular injection.

39. The method of any one of claims 31 to 37, wherein the antibody is expressed in the muscle cell and released into the circulation of the subject.

40. The method of claim 39, further comprising monitoring the presence of the antibody in the circulation at least once.

41. A method for treating or preventing an inflammatory or autoimmune disease or disorder, comprising administering an effective amount of the composition of claim 6 to a subject in need thereof.

42. The method of claim 41, wherein the antibody targets and neutralizes the action of IL-23.

43. The method of claim 42, wherein the subject has plaque psoriasis or Crohn's disease.

44. The method of any one of claims 41 to 43, wherein the DNA construct is according to any one of claims 19 to 24.

45. The method of claim 41, wherein the subject has arthritis.

46. The method of claim 45, wherein the therapeutic protein is an antibody that binds to and neutralizes TNF-α or IL-6.

47. The method of claim 45 or 46, wherein the DNA construct is according to any of claims 8 to 18.

48. The method of any one of claims 41 to 47, wherein administering comprises injection and electroporation.

49. The method of any one of claims 41 to 48, wherein the administering is intramuscular injection.

50. The method of any one of claims 41 to 49, wherein the antibody is expressed in the muscle cell and released into the circulation of the subject.

51. The method of claim 50, further comprising monitoring the presence of the antibody in the circulation at least once.

52. The method for treating or preventing cancer, comprising administering an effective amount of the DNA composition of claim 4 to a subject in need thereof.

53. The method of claim 52, wherein the subject has cancer, which is optionally stage 1, stage 2, stage 3, or stage 4.

54. The method of claim 53, wherein the cancer is a solid tumor.

55. The method of claim 54, wherein the DNA construct is administered following tumor resection.

56. The method of claim 52, wherein the DNA construct is administered with or following chemotherapy, radiation therapy, or cell therapy that is optionally a T cell therapy.

57. The method of claim 56, wherein the cancer is a blood cancer.

58. The method of any one of claims 52 to 57, wherein the DNA construct is according to any of claims 8 to 18.

59. The method of any one of claims 52 to 58, wherein administering comprises injection and electroporation.

60. The method of any one of claims 52 to 59, wherein the administering is intramuscular injection.

61. The method of any one of claims 52 to 60, wherein the antibody is expressed in the muscle cell and released into the circulation of the subject.

62. The method of claim 61, further comprising monitoring the presence of the antibody in the circulation at least once.

63. A method for treating or preventing an inflammatory eye disease, comprising administering an effective amount of the composition of claim 3.

64. The method of claim 63, wherein the DNA construct is according to any of claims 8 to 18.

65. The method of claim 63 or 64, wherein administering comprises injection and electroporation.

66. The method of claim 65, wherein the administering is intramuscular injection.

67. The method of claim 66, wherein the antibody is expressed in the muscle cell and released into the circulation of the subject.

68. The method of claim 67, further comprising monitoring the presence of the antibody in the circulation at least once.

69. The method of any one of claims 63 to 68, wherein the inflammatory eye disease is selected from an inflammatory eye disease associated with corneal transplant, diabetic macular edema, diabetic retinopathy, dry eye disease, scleritis, blepharitis, keratitis, conjunctivitis, chorioretinal inflammation, chorioretinitis, iridocyclitis, iritis, posterior cyclitis, and uveitis.

70. The method of claim 69, wherein the inflammatory eye disease is associated with a corneal allograft, or a corneal allograft rejection.

71. The method of claim 69, wherein the inflammatory eye disease is uveitis which is anterior uveitis, panuveitis, intermediate uveitis or posterior uveitis.

72. A method for improving a patient response to allogeneic hematopoietic stem cell transplantation (aHSCT), comprising administering an effective amount of the composition of claim 3 to a subject in need, and wherein the therapeutic agent binds to and neutralizes a pro-inflammatory cytokine.

73. The method of claim 72, wherein the DNA construct is according to any of claims 8 to 18.

74. The method of claim 72 or 73, wherein administering comprises injection and electroporation.

75. The method of claim 74, wherein the administering is intramuscular injection.

76. The method of claim 75, wherein the antibody is expressed in the muscle cell and released into the circulation of the subject.

77. The method of claim 76, further comprising monitoring the presence of the antibody in the circulation at least once.

78. A method for treating chronic neutropenia, comprising administering an effective amount of the composition of claim 3 to a subject in need, wherein the DNA construct expresses G-CSF.

79. The method of claim 78, wherein the DNA construct is according to any of claims 8 to 18.

80. The method of claim 78 or 79, wherein administering comprises injection and electroporation.

81. The method of claim 80, wherein the administering is intramuscular injection.

82. The method of claim 81, wherein the antibody is expressed in the muscle cell and released into the circulation of the subject.

83. The method of claim 82, further comprising monitoring the presence of the antibody in the circulation at least once.

84. A method for treating anemia, comprising administering an effective amount of the composition of claim 3 to a subject in need, wherein the DNA construct expresses erythropoietin.

85. The method of claim 84, wherein the DNA construct is according to any of claims 8 to 18.

86. The method of claim 84 or 85, wherein administering comprises injection and electroporation.

87. The method of claim 86, wherein the administering is intramuscular injection.

88. The method of claim 87, wherein the antibody is expressed in the muscle cell and released into the circulation of the subject.

89. The method of claim 88, further comprising monitoring the presence of the antibody in the circulation at least once.

90. A method for treating a rare disease, comprising administering an effective amount of the composition of claim 3 any to a subject in need thereof.

91. The method of claim 90, wherein the rare disease is an enzyme deficiency, and the DNA construct provides enzyme replacement therapy.

92. The method of claim 90 or 91, wherein the DNA construct is according to any of claims 8 to 18.

93. The method of any one of claims 90 to 92, wherein administering comprises injection and electroporation.

94. The method of claim 93, wherein the administering is intramuscular injection.

95. The method of claim 94, wherein the antibody is expressed in the muscle cell and released into the circulation of the subject.

96. The method of claim 95, further comprising monitoring the presence of the antibody in the circulation at least once.


SEQUENCE LISTING (27 sequences)

SEQ ID NO: 1 - DNA, 2259 bp, synthetic construct
  source 1..2259: mol_type = other DNA
attggctattggccaactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtcgctagcctcgagagatcacttctggctaataaaagatcagagctctagagatctgtgtgttggttttttgtggatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcagcacagcaagggggaggattgggaagacaatagcaggcatgctggggatgcggtgggctctatgggtacctctctctctctctctctctctctctctctctctctctctcggtacctctctctctctctctctctctctctctctctctctctctcggtaccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatt

SEQ ID NO: 2 - DNA, 52 bp, synthetic construct
  source 1..52: mol_type = other DNA
tcttttctgcagtcaccgtcgctagcctcgagagatcacttctggctaataa

SEQ ID NO: 3 - DNA, 1564 bp, synthetic construct
  source 1..1564: mol_type = other DNA
attggctattggccaactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgccgcggccgggaacggtgcattggaacgcggattccccgtgccaagagtgacgtaagtaccgcctatagactctataggcacacccctttggctcttatgcatctcgttgctgccgcgcgcgccaccagacataatcgctgacacactgacagactgttcctttcctttttttttttttgcagtcaccgtcgctagcctcgagagatcacttctggctaataaaagatcagagctctagagatctgtgtgttggttttttgtggatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcagcacagcaagggggaggattgggaagacaatagcaggcatgctggggatgcggtgggctctatgggtacctctctctctctctctctctctctctctctctctctctctcggtacctctctctctctctctctctctctctctctctctctctctcggtaccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatt

SEQ ID NO: 4 - DNA, 52 bp, synthetic construct
  source 1..52: mol_type = other DNA
ttttttttgcagtcaccgtcgctagcctcgagagatcacttctggctaataa

SEQ ID NO: 5 - DNA, 2337 bp, synthetic construct
  source 1..2337: mol_type = other DNA
attggctattggccaactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtcgctagcaggagaaagagaggatccagcggcgaaggcagaggcagccttcttacatgtggcgacgtggaagagaaccccggacctctcgagagatcacttctggctaataaaagatcagagctctagagatctgtgtgttggttttttgtggatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcagcacagcaagggggaggattgggaagacaatagcaggcatgctggggatgcggtgggctctatgggtacctctctctctctctctctctctctctctctctctctctctcggtacctctctctctctctctctctctctctctctctctctctctcggtaccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatt

SEQ ID NO: 6 - DNA, 46 bp, synthetic construct
  source 1..46: mol_type = other DNA
tcttttctgcagtcaccgtcgctagcaggagaaagagaggatccag

SEQ ID NO: 7 - DNA, 45 bp, synthetic construct
  source 1..45: mol_type = other DNA
ggaagagaaccccggacctctcgagagatcacttctggctaataa

SEQ ID NO: 8 - DNA, 2340 bp, synthetic construct
  source 1..2340: mol_type = other DNA
attggctattggccaactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtcgctagcaggagaaagagaggatccagcggcgccaccaacttcagcctgcttaaacaggcaggcgacgtggaagagaaccccggacctctcgagagatcacttctggctaataaaagatcagagctctagagatctgtgtgttggttttttgtggatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcagcacagcaagggggaggattgggaagacaatagcaggcatgctggggatgcggtgggctctatgggtacctctctctctctctctctctctctctctctctctctctctcggtacctctctctctctctctctctctctctctctctctctctctcggtaccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatt

SEQ ID NO: 9 - DNA, 46 bp, synthetic construct; identical to SEQ ID NO: 6
  source 1..46: mol_type = other DNA

SEQ ID NO: 10 - DNA, 46 bp, synthetic construct
  source 1..46: mol_type = other DNA
tggaagagaaccccggacctctcgagagatcacttctggctaataa

SEQ ID NO: 11 - protein, 466 aa, synthetic construct
  REGION 1..466: note = Synthetic Sequence
MDWTWRVFCLLAVAPGAHSEVQLVQSGAEVKKPGESLKISCKGSGYSFSNYWIGWVRQMPGKGLEWMGIIDPSNSYTRYSPSFQGQVTISADKSISTAYLQWSSLKASDTAMYYCARWYYKPFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 238 aa, synthetic construct
  REGION 1..238: note = Synthetic Sequence
MVLQTQVFISLLLWISGAYGQSVLTQPPSVSGAPGQRVTISCTGSSSNIGSGYDVHWYQQLPGTAPKLLIYGNSKRPSGVPDRFSGSKSGTSASLAITGLQSEDEADYYCASWTDGLSLVVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 730 aa, synthetic construct
  REGION 1..730: note = Synthetic Sequence
MDWTWRVFCLLAVAPGAHSEVQLVQSGAEVKKPGESLKISCKGSGYSFSNYWIGWVRQMPGKGLEWMGIIDPSNSYTRYSPSFQGQVTISADKSISTAYLQWSSLKASDTAMYYCARWYYKPFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRRKRGSSGEGRGSLLTCGDVEENPGPMVLQTQVFISLLLWISGAYGQSVLTQPPSVSGAPGQRVTISCTGSSSNIGSGYDVHWYQQLPGTAPKLLIYGNSKRPSGVPDRFSGSKSGTSASLAITGLQSEDEADYYCASWTDGLSLVVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 731 aa, synthetic construct
  REGION 1..731: note = Synthetic Sequence
MDWTWRVFCLLAVAPGAHSEVQLVQSGAEVKKPGESLKISCKGSGYSFSNYWIGWVRQMPGKGLEWMGIIDPSNSYTRYSPSFQGQVTISADKSISTAYLQWSSLKASDTAMYYCARWYYKPFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRRKRGSSGATNFSLLKQAGDVEENPGPMVLQTQVFISLLLWISGAYGQSVLTQPPSVSGAPGQRVTISCTGSSSNIGSGYDVHWYQQLPGTAPKLLIYGNSKRPSGVPDRFSGSKSGTSASLAITGLQSEDEADYYCASWTDGLSLVVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 468 aa, synthetic construct
  REGION 1..468: note = Synthetic Sequence
MDWTWRVFCLLAVAPGAHSEVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 235 aa, synthetic construct
  REGION 1..235: note = Synthetic Sequence
MVLQTQVFISLLLWISGAYGDIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIKRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 729 aa, synthetic construct
  REGION 1..729: note = Synthetic Sequence
MDWTWRVFCLLAVAPGAHSEVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRRKRGSSGEGRGSLLTCGDVEENPGPMVLQTQVFISLLLWISGAYGDIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIKRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 730 aa, synthetic construct
  REGION 1..730: note = Synthetic Sequence
MDWTWRVFCLLAVAPGAHSEVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRRKRGSSGATNFSLLKQAGDVEENPGPMVLQTQVFISLLLWISGAYGDIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIKRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 469 aa, synthetic construct
  REGION 1..469: note = Synthetic Sequence
MDWTWRVFCLLAVAPGAHSQVQLVQSGAEVKKPGSSVKVSCKASGYTFTDQTIHWMRQAPGQGLEWIGYIYPRDDSPKYNENFKGKVTITADKSTSTAYMELSSLRSEDTAVYYCAIPDRSGYAWFIYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 234 aa, synthetic construct
  REGION 1..234: note = Synthetic Sequence
MVLQTQVFISLLLWISGAYGDIQMTQSPSSLSASVGDRVTITCKASRDVAIAVAWYQQKPGKVPKLLIYWASTRHTGVPSRFSGSGSRTDFTLTISSLQPEDVADYFCHQYSSYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 729 aa, synthetic construct
  REGION 1..729: note = Synthetic Sequence
MDWTWRVFCLLAVAPGAHSQVQLVQSGAEVKKPGSSVKVSCKASGYTFTDQTIHWMRQAPGQGLEWIGYIYPRDDSPKYNENFKGKVTITADKSTSTAYMELSSLRSEDTAVYYCAIPDRSGYAWFIYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRRKRGSSGEGRGSLLTCGDVEENPGPMVLQTQVFISLLLWISGAYGDIQMTQSPSSLSASVGDRVTITCKASRDVAIAVAWYQQKPGKVPKLLIYWASTRHTGVPSRFSGSGSRTDFTLTISSLQPEDVADYFCHQYSSYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 730 aa, synthetic construct
  REGION 1..730: note = Synthetic Sequence
MDWTWRVFCLLAVAPGAHSQVQLVQSGAEVKKPGSSVKVSCKASGYTFTDQTIHWMRQAPGQGLEWIGYIYPRDDSPKYNENFKGKVTITADKSTSTAYMELSSLRSEDTAVYYCAIPDRSGYAWFIYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRRKRGSSGATNFSLLKQAGDVEENPGPMVLQTQVFISLLLWISGAYGDIQMTQSPSSLSASVGDRVTITCKASRDVAIAVAWYQQKPGKVPKLLIYWASTRHTGVPSRFSGSGSRTDFTLTISSLQPEDVADYFCHQYSSYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 204 aa, synthetic construct
  REGION 1..204: note = Synthetic Sequence
MAGPATQSPMKLMALQLLLWHSALWTVQEATPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 24 - protein, 226 aa, synthetic construct
  REGION 1..226: note = Synthetic Sequence
MAGPATQSPMKLMALQLLLWHSALWTVQEATPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQPGGGGSHHHHHHVSGWRLFKKIS

SEQ ID NO: 25 - protein, 390 aa, synthetic construct
  REGION 1..390: note = Synthetic Sequence
MAGPATQSPMKLMALQLLLWHSALWTVQEATPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQPGGGGGSGGGGSGGGGSVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 26 - protein, 215 aa, synthetic construct
  REGION 1..215: note = Synthetic Sequence
MAGPATQSPMKLMALQLLLWHSALWTVQEATPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQPGGGGSHHHHHH

SEQ ID NO: 27 - protein, 220 aa, synthetic construct
  REGION 1..220: note = Synthetic Sequence
MAGPATQSPMKLMALQLLLWHSALWTVQEATPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQPGGGGSVSGWRLFKKIS